(19) United States
(12) Patent Application Publication
(10) Pub. No.: US 2014/0190481 A1
(43) Pub. Date: Jul. 10, 2014

(54) Acoustic Ventilation and Respiratory Booster Machine

(71) Applicant: Mohammad R. JAM, Riverside, CA (US)

(72) Inventor: Mohammad R. JAM, Riverside, CA (US)

(21) Appl. No.: 14/135,995

(22) Filed: Dec. 20, 2013

Related U.S. Application Data
(60) Provisional application No. 61/748,798, filed on Jan. 4, 2013.

(51) Int. Cl. A61M 16/00; A61M 16/06; A61M 16/20; A61M 16/10; A61M 16/04; A61M 16/08

(52) U.S. Cl. CPC A61M 16/0006; A61M 16/0486; A61M 16/0883; A61M 16/0069; A61M 16/20; A61M 16/10; A61M 16/0666; A61M 16/0051
USPC 128/203.12; 128/204.18; 128/204.23; 128/205.24

(57) ABSTRACT

An acoustic ventilator using feedback control is designed to generate and exert a desired mixture of pressure oscillations into a supply of gas entering a subject's airways to maintain optimal ventilation and perfusion in the lungs. According to the subject's biological specifications and real-time medical condition, computers and human interface control the vibrations in intensity and frequency along with the pressure and composition of blended gases in order to enhance oxygenation and CO₂ clearance. Acoustic ventilation will cause the ventilating gases to diffuse through the subject's lungs without the aid of a separate ventilator, and with or without spontaneous breathing.

### CROSS REFERENCE TO RELATED APPLICATION

This application claims the benefit of Provisional 61/748,798 filed Jan. 4, 2013 the entire contents of which is hereby expressly incorporated by reference herein.

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC

Not Applicable

### BACKGROUND OF THE INVENTION

1. Field of the Invention

This invention relates to improvements in technologies and procedures involved in artificial ventilation of lungs of humans and/or animals. More particularly, the present acoustic ventilation (AV) combines sound energy from electro-acoustic transducer(s) in a multi-modal respiratory ventilation system to enhance gas exchange by improving alveolar ventilation and potentiating gas diffusion. Acoustic ventilation can maintain satisfactory gas exchange with lower mean airway pressures (Pmaw) and less fraction of inhaled oxygen (FiO₂). It can also facilitate pulmonary toilet, and assist with administration of aerosolized medicines.

2. Description of Related Art including information disclosed under 37 CFR 1.97 and 1.98

The oldest method of ventilating lungs was by the use of blacksmiths bellows. It was imprecise and operator dependent and at times dangerous. This method lasted for a long time till it was replaced with iron lungs which saved many lives during polio epidemics. Iron lungs were bulky and difficult to adjust and now are rarely used. A newer version of these machines called biphasic cuirass ventilators (BCV) is utilized nowadays in certain conditions. It uses a shell around the patient's chest instead of the tank and exerts negative and positive extra-thoracic pressures in order to pull the air and out of the lungs.

More manageable machines using positive pressure ventilation (PPV) were introduced and became popular for their precision and ease of control. Conventional mechanical ventilators (CMV) mimic regular breathing patterns by pushing a desirable column of gas into a subject's airways at tidal volumes close to normal breath volumes, and in rates similar to the subject's breath rate. These machines can be fine-tuned to the needs of the patient by adjustments on fraction of inspired oxygen (FiO₂), inspiration and expiration timing, and volume/pressure limit settings, etc. A typical CMV does not use negative pressure during exhalation and instead, relies on the tendency of lungs to recoil and deflate spontaneously. Re-inflation of collapsed alveoli needs high pressures which can be damaging to lungs. In order to prevent this, some residual airway pressure called positive end expiratory pressure (PEEP) is maintained. CMVs usually have an upper limit of 60-90 breaths per minute.

An interesting method of assisted ventilation in neonates and premature babies is called bubble CPAP which is still in use in some medical centers despite its inherent imprecision. This method follows stochastic rules of ventilation and hence supplies effective alveolar ventilation to various parts of the lungs.

High frequency oscillatory ventilators (HFV, HFO, HFOV) were developed to benefit patients who are not stable on CMV. These machines provide some benefits in certain conditions such as pulmonary interstitial emphysema PIE, pneumothorax, and possibly respiratory distress syndrome RDS. They use much higher respiratory rates, up to 900/min or 15 Hz, with remarkably lower tidal volumes. Other types of HFV are high frequency jet ventilators (HFJV), High Frequency Percussive Ventilator (HFPV), high frequency fan ventilator (HFFV), high frequency flow interruption (HFFI), etc. Some other machines use combination of CMV and HFOV such as Infant Star/Adult Star™. They are collectively called high frequency ventilators HFVs in the art.

The highest oscillatory-rate setting on any HFV in the industry today is 50-100 Hz although most of these machines operate best below 15-20 Hz. There are at least two plausible explanations why rates higher than 15-20 Hz are not usable in these machines. One reason being extremely loud operational noise created after their oscillation frequencies reach audible range of >20 Hz, and the other even more important reason is development of ventilator choking phenomenon in higher rates secondary to ET tube attenuation of pressure oscillations due to compressibility of gases, i.e. the very gas about to enter the ET tube will be pulled back by the oscillator before it gets a chance to move into the patient airways resulting in a sheer drop in the ventilator's gas diffusion capacity. In some HFVs there are even more issues at higher frequencies as oscillation amplitude of their diaphragm/bellows will be attenuated at frequencies higher than 10-15 Hz due to slew rate limit, i.e. the transit time of their oscillator would become greater than the cycle time required by the frequency adjustment.

Most HFVs have to generate oscillatory pressure waves (δp) of 80-90 cmH₂O or even higher at attachment point of their patient circuit to ET tube in order to create pressure oscillations of only 7-8 cmH₂O in the trachea. These numbers change according to alterations made in the power (swinging range of the oscillator), ET tube diameter, frequency, Pmaw, compliance, and inspiratory time or I/E ratio. Obviously, the amount of loss will be greater in higher frequencies and with smaller ET tubes. Endotracheal tube and lung impedance drastically attenuate higher frequency pressure waves of these machines and even distort their waveform. In a HFOV model (Carefusion 3100) about 90% of the oscillatory pressure passing a 2.5 mm ET tube is lost at 15 Hz with compliance of 1 ml/cmH₂O and inspiratory time of 33%, according to its operation manual. All of these ventilators are practically useless in the very beneficial sonic ranges of above 20 Hz. At the present time there are no ventilator machines operating competently in frequency ranges above 15-20 Hz although some of them might have adjustment settings of up to 50-100 Hz.

In acoustic or sonic ventilation, the method of current invention, well-tolerated higher frequencies can be employed and used for the benefit of patients while impedance of even the smallest ET tubes can be easily overcome with focused and guided sonic energy. Current invention is able to rapidly diffuse fresh gases deep down the smallest end-bronchioles by the use of a specially designed and innovative tube system called acoustic/sonic flue. This level of prompt and profound pulmonary gas dispersion, dissemination, and diffusion has never been achieved before.

### BRIEF SUMMARY OF THE INVENTION

Ventilation of lungs through positive airway pressure is not natural and can harm the lungs. Main factors anticipating the extent of ventilator induced lung injury (VILI) are peak inspiratory pressure (PIP), mean air way pressure (Mpaw), duration of artificial ventilation, and fraction of inhaled oxygen (Fio²). Any effort in reducing harm instigated by the above mentioned parameters is crucially important in the art of artificial ventilation. [1]

Almost all contemporary ventilation techniques rely on positive airway pressure (PPV) except biphasic cuirass ventilator (BCV) which is still not exempt it terms of VILI. Years ago, multiple various techniques of PPV with high frequency oscillations (HFO) emerged and were initially hyped as deemed less injurious to the lungs and helpful in certain conditions, particularly in neonates. In order to create needed pressure oscillations (Sp) at the distal end of the ET tube or in the patient's airways, these machines have to generate high amplitude pressure fluctuations, as high as 80-90 cmH₂O or even higher, in a monotone yet adjustable high frequency rate mainly in the range of 180-900 oscillations per minute or 3-15 Hz. Because of compressibility of gases and ET tube attenuation of pressure oscillations, effective ventilatory command of these machines begin to diminish when rate go over 10-15 Hz. Although higher oscillatory rates are advantageous and desirable in certain condition especially in premature newborns but because of above mentioned limiting factors, high frequency ventilators of any type have continually failed to produce any satisfactory ventilatory capacity above 15-20 Hz. A sheer drop in airway Sp and alveolar ventilation is witnessed when these machines struggle dealing with low pulmonary compliance, narrow ET tube, and rates above 10 Hz. At times, healthcare providers have to lower frequencies down to 5-8 Hz in order to maintain satisfactory Sp in patient airways while higher rates are indeed needed to ventilate a consolidated, wet, low compliant lung. This is an example of limitation of a therapeutic measure due to technological complications. Pulmonary standard of care can enhance if higher oscillatory levels could be accomplished by overcoming such technical complications. Acoustic ventilator has easily overcome said frequency rate limitation.

Variable rhythms and rates of pressure oscillations, or pulsations, play vital roles throughout our body. They are slow in our gastrointestinal tract and faster in our cardiovascular system for example. Our respiratory system operates under two different rhythms, a slower pressure oscillation of about 10-40 per min which is regular breathing, and a faster set of pressure vibrations mainly in the range of 50-500 Hz made by the larynx at times of talking, singing, humming, grunting, crying, screaming, growling, etc. These sonic vibrations boost respiration at times of need in form of moaning or grunting in sickness, and growling or screaming in fight and fear, for example. Even expectoration is dependent on sound energy as coughing and clearing our throat is helped by these fast-paced vibrations. If not limited by technology, ventilation of lungs with added oscillatory rates in sonic range is physiologically very desirable indeed. Advantages of sonic ventilation are even more pronounced when we face pathology particularly in the very young and vulnerable lungs of a newborn, although these machines are proficient in ventilating lungs of even the largest animals.

In order to comprehend how sound energy can assist respiration and expectoration one has to study physics of sound, non-Newtonian fluids, and cymatics. Physics of sound tells us how sound energy can move/vibrate/resonate molecules and how we can manage transmission of gases in non-tubular and tubular structures such as ducts, waveguides, or our airways. Cymatics in its newly extended perspective is the study of energy dispersion in various sound frequencies and harmonics in gas, liquid, and solid media. Non-Newtonian fluids are viscous liquids that behave differently from Newtonian or inviscid fluids. These liquids change their viscosity rapidly and temporarily when exposed to impact or vibrations. Examples of non-Newtonian fluids in our body would be saliva, sputum, lymph, blood, phlegm, etc. Nature uses effects of sound energy on viscid fluids to mobilize mucus in our lungs, now try to clear your throat without making anysound!

Sonic vibrations created by vocal cords in larynx and amplified in upper airway compartments such as pharynx, oral cavity, nasal passages, and paranasal sinuses resonate the lungs and make alveoli tremble. Paranasal sinuses and nasopharynx are parts of the natural sonic apparatus helping respirations as they act like the hollow body of a guitar or violin where sonic vibrations get amplified. These oscillations will practically increase effective gas exchange surface area by shaking alveoli while facilitating diffusion of gases inside even the smallest airways and across alveolar membrane. A simple example of this phenomenon is facilitated passage of flour particles through a sifter/sieve when it is shaken. We grunt in sickness or growl in danger not only to increase airway pressure and recruit more alveoli but also to shake them and ease gas diffusion. Nature created our larynx and nasopharynx as integral parts of our pulmonary system to bestow a vital functional reserve, a marvel of physiologic organ harmony, or should we say harmonic organ physiology.

In our lungs, these fast paced pressure oscillations are helpful in keeping the alveoli open and get the secretions out. When we intubate a patient in order to ventilate their lungs we disable their natural sonic ability to boost respirations and maintain expectoration through coughing, grunting, and moaning. Acoustic ventilation (AV), also named sonic ventilation (SV) or sound bloc ventilation (SBV), can give this eliminated natural ability back to intubated patients in operating rooms, intensive care units, and in other situations. Unfortunately sonic respiratory rates, or subsonic rates with added sonic energy, have not been systematically studied and currently there are no ventilator machines in the market capable of effectively ventilating lungs in acoustic range. Results of preliminary in-vitro and in-vivo studies with sound bloc ventilation, started in 2002 by the inventor in Ontario, Canada have clearly been indicative of evolutionary properties of sonic oscillations in the art of ventilation.

Currently, there are no machines in the field using variable combination of pressure oscillations in the manner applied in AV/SV/SBV which are generally capable of producing pressure oscillations ranging from 6 times per minute (0.1 Hz) all the way up to 120 million times per minute (2 MHz). These pressure oscillations can range from negative pressures down to minus 10-20 cm H₂O and up to 100 cm H₂O. Although power intensity of acoustic ventilator machine AVM can range from zero up to a whopping 150 dB inside the machine and airways but its operational noise remains well within acceptable regulated range. The unique, innovative, and useful feature of this invention is simulating nature, at times inevitably exaggerated, by generating and transferring bundled sonic pressure oscillations into a patient's airways. Indeed, AV/SV/SBV can deliver pressure oscillations of 1 Hz-20 KHz with or without vibrations of 20 KHz-2 MHz on the background of airway pressure variations of 0-60 cmH₂O at a rate of 0-60 undulations per minute. Compared with all other known methods of ventilation, this is the most efficient and safest way we can ventilate lungs. In certain medical conditions negative airway pressures of up to −20 cmH₂O, or even higher, are transiently employed by the machine to help with extraction of pulmonary secretions.

As mentioned earlier, the highest useful pressure oscillation rate in currently available ventilators is about 15-20 Hz, and not to be limited by theory, there are at least two plausible reasons for that. First, significantly louder operational noise when oscillations get close to audible range above 1200/min or 20 Hz, and second or the main reason is the ET tube attenuation or the “choking phenomenon” which begins to show effect at around 10-15 Hz in some high frequency oscillatory ventilator (HFOV) assemblies. Chocking phenomenon happens because higher rates will pull back the very air about to enter an ET tube before it has a chance to cross the length of the tube. It happens because gases are naturally compressible. In some HFOV machines such as CareFusion's 31 OOA and 31008, the oscillator's response time might not even be sufficient to accommodate higher frequencies, according to their operation manual.

Choking/jamming phenomenon does not happen with AV/SV/SBV even at frequencies above 50 Hz because of its unique and innovative waveguide called sonic flue which is able to create a high velocity gas flow, back and forth through even the narrowest ET tubes. Sonic Flue, the specially designed waveguide and a major claim of current invention, has varied and multiple functional models. These waveguides are also interchangeably called acoustic flue, acoustic/sonic waveguide, acoustic or sonic funnel, acoustic or sonic accelerator, acoustic/sonic spiral/helix, infrasonic flue/waveguide, ultrasonic flue/waveguide, infrasonic/ultrasonic funnel, and other similar names in this document. It is strange that up until now there has been no useful application for such valuable narrowing tubular reflective physical barrier of infrasonic/ultrasonic/sonic waves.

Conventional ventilation methods of CMV, HFV, and even BCV will damage delicate airways and disrupt alveolar scaffolding, at times irreversibly, when used over a span of hours or days. Alveolar ventilation in these machines is dependent on flow and diffusion of gases in the airways instigated by pressure oscillations from above with some penetrance into the smaller airways. Alveolar ventilation is exponentially enhanced when flow of gases and their rapid diffusion down to the end-bronchioles is charged with sonic energy.

Other well-known side effects of contemporary methods of ventilation are under/over ventilation, lung over-distention, air leak, pulmonary interstitial emphysema (PIE), pneumothorax and pneumopericardium, pneumomediastinum, pneumoperitoneum, bronchopulmonary dysplasia (BPD) and chronic lung disease (CLD), necrotizing tracheal bronchitis (NTB), atelectasis, mucus plugging of the endotracheal tube or airways, bradycardia, and hypotension. Secondary implications of ventilation with currently available technologies are intraventricular hemorrhage (IVH) and cerebral palsy (CP) with permanent global developmental delays. Incidence of these and other ventilator associated complications will diminish in future when AV/SV/SBV becomes standard of care.

Various objects, features, aspects, and advantages of the present invention will become more apparent from the following detailed description of preferred embodiments of the invention, along with the accompanying drawings in which like numerals represent like components.

### BRIEF DESCRIPTION OF THE SEVERAL VIEWS OF THE DRAWING(S)

FIG. 1 shows a schematic presentation of sonic flue and sonic waveguides.

FIG. 2 shows some schematic presentations of various elongated sonic waveguide funnels.

FIG. 3 shows a schematic presentation of how sonic waveguides work.

FIG. 4 shows a schematic presentation of helical and maze waveguides.

FIG. 5 shows a schematic presentation of sonic waveguide gas wash out, and air-hub.

FIG. 6 shows a schematic presentation of sonic waveguide generated with electronic signals.

FIG. 7 shows a schematic presentation of acoustic ventilation with diagnostic and interventional acoustic signal analyzer.

FIG. 8 shows a schematic presentation of acoustic ventilation with an altered patient circuit/air-hub.

FIG. 9 shows a schematic presentation of various noise cancellation techniques.

FIG. 10 shows a schematic presentation of acoustic ventilation in harmony with other ventilation systems.

FIG. 11 shows a schematic presentation of double barrel acoustic ventilation.

FIG. 12 shows a schematic presentation of double barrel ET tubes.

### DETAILED DESCRIPTION OF THE INVENTION

### Brief Description

Physical design of HFOV machines gets them into a functional limitation in higher rates in a process to be called HFV choking phenomenon. Alveolar ventilation and CO₂ clearance in HFV depends on Sp in patient airways. In difficult cases of CO₂ retention such as RDS, these machines easily reach their highest power limit after which point the only way to improve alveolar ventilation would be to lower their oscillatory rates which is not desirable. Presently, there are no ventilator machines capable of sufficiently defying dire flow resistance of an ET tube confronting a low pulmonary compliance, a case of technological restraint contributing to high mortality and morbidity rates of certain ailments such as RDS.

Current invention describes a new method of ventilation of lungs in a safer meanwhile more effective and natural manner. It generates variable blocs of infrasonic, sonic, and ultrasonic pressure oscillations, ranging from 0-150 dB in power intensity, and delivers them into a subject's lungs through a specially designed tubing system encasing a column of blended desired gases destined to enter a subject's airways. The machine can also work like a CMV machine at the same time by exerting variable positive and negative pressure into said column of blended gases at a rate of 0-60/min with pressure undulations from −10 cmH₂O to +60 cmH₂O (usually from +3 cmH₂O to +15 cmH₂O under normal conditions or mild pathology). In this manner, while the trachea and main bronchi are being ventilated in a fashion like CMV machines, all possible subsonic, sonic, and ultrasonic pulmonary resonance rates are instigated and used for the benefit of the patient. Indeed, acoustic/sonic ventilator is simply a combined CMV and HFOV with added sonic & ultrasonic vibrations. Negative pressures are only used briefly and randomly with sudden and abrupt pressure plunges in order to facilitate clearance of airway secretions. Such abrupt negative airway pressure exertions, to be discussed in detail later, are normally triggered by acoustic ventilator right after a full inspiratory period.

Sonic vibrations travel to end bronchioles with the speed of sound, literally, and make alveolar membranes quiver and therefore, increase effective gas exchange surface area and facilitate diffusion of gases across the membranes. According to a subject's age, physical characteristics, and real-time medical condition(s), human interface and/or automated feedback control will adjust frequency blocs, pressures, and FiO² to maintain optimal gas exchange with least harm to lungs. Said vibration blocs and their specially designed variations, usually moving from lower pitch harmonics to higher ones, also promote pulmonary toilet by process of sonic wave nodal displacement, as explained with physics of sound and cymatics.

In HFOV, pulmonary resonance is typically achieved with rates of 3-8 Hz in adults and 8-15 in children and infants. As a general rule, larger lungs resonate better in lower frequencies compared to smaller ones which resonate better in higher frequencies. One can actually observe the resonance by visualization of fast vibrations of the patient's chest. There is at least one more pulmonary resonance, called acoustic resonance, which is not visible but can be felt by our tactile sensation and heard by our ears. Acoustic resonance happens normally when we talk, sing, grunt, or growl. Unfortunately, this level of pulmonary resonance and its spectacular health benefits have been completely ignored by all ventilation technologies up until now and subsequently, patients and physicians have lost so many battles in so many cases and for so many years.

Normally, acoustic resonance of smaller lungs/airways is actuated in higher frequencies, and vice versa. Acoustic resonance in human lungs ranges between 80-500 Hz which is the frequency of human voice, clearly above the range that current ventilation technologies can perform. This naturally occurring resonance, not previously used in the industry, is one of the main objects of the current invention to be employed in order to enhance gas exchange, facilitate pulmonary toilet, and reduce VILI. Resonance of lungs at cellular or molecular levels in even higher frequencies is another object of this invention.

Use of sonic vibrations can be very beneficial in some acute and chronic respiratory disorders such as respiratory distress syndrome (RDS/HMD), pulmonary edema of any cause, chronic obstructive pulmonary disease (COPD), pneumonia, asthma, pulmonary hemorrhage, pulmonary atelectasis, pulmonary embolism, malignancies, bronchiectasis, cystic fibrosis CF, bronchopulmonary dysplasia BPD and chronic lung disease CLD, interstitial lung disease and pulmonary fibrosis, respiratory insufficiency due to central or peripheral nervous system such as cerebral palsy CP or spinal muscular atrophy and some other neurological disorders, among many other pulmonary conditions.

A description of similar ventilation system has been mentioned in the inventor's patented device called Respiratory Booster Machine RBM (Patent U.S. Pat. No. 7,478,634 January 2009, application Ser. No. 10/654,816 Filed Sep. 3, 2003). Various improvements to RBM and its use as an independent ventilator machine are explained herein. Acoustic ventilation and RBM increase the effective alveolar surface area through vibrating the alveolar membranes and therefore, augment gas diffusion and gas exchange through this novel effect which is lacking in all other ventilator machines currently available.

Sonic ventilation (SV) can reduce progression of neonatal chronic lung disease as it enables us to ventilate lungs sufficiently using lower air way pressure (Paw) and less oxygen (Fio²), compared with currently available techniques. Sonic ventilation exerts less traction forces onto the airways and has the ability to keep micro spaces open and allow better alveolar formation. It may improve vascular and capillary development which is crucial in growth of healthy lung tissue.

Another advantage of AV is improved dispersion of surfactant into end bronchioles and alveolar spaces with faster opening of alveoli and elimination of need to use higher pressures. In this way, AV/SV/SBV can reduce incidence of bronchopulmonary dysplasia BPD, chronic lung disease CLD, retinopathy of prematurity ROP, hypoxic ischemic encephalopathy HIE, and cerebral or intraventricular hemorrhage IVH, among other conditions in neonates. Intermittent or continuous use of AV in first few hours or days of life in premature babies may promote development of more intricate alveolar scaffolding and prevent apoptosis of alveolar sac lining cells. The level and degree of such developmental effects of sonic vibrations on lung tissue is a subject of future studies.

In 2002, multiple working prototypes utilizing electro-acoustic transducers of coil and magnet, piezoelectric, and magnetorestrictive technology were made for the first time by the inventor in Ontario, Canada. Preliminary tests with the transducers in the above mentioned bundled frequencies were performed and parameters such as pressure changes Op, flow, tidal volume Vt, and minute volume Vm were assessed. Subsequently, respiratory booster machine RBM was developed and a patent application was filed with USPTO in 2003 which resulted in a patent granted in 2009. In April of 2013, method of acoustic ventilation and results of successful sonic ventilation of a rat under anesthesia was formally disclosed during a 40 minute slide presentation by the inventor at the International Conference of High Frequency Oscillatory Ventilation to an audience of healthcare providers, professionals, and experts in the field. Scientists and delegates from multiple U.S. and foreign ventilator manufacturing corporations were present as well.

Various objects, features, aspects, and advantages of the present invention will become more apparent from the following detailed description of preferred embodiments of the invention, along with the accompanying drawings in which like numerals represent like components.

### DETAILED DESCRIPTION

This document describes a new and improved technique of ventilation of lungs, achieved by merging various pressure waves with employment of substantially higher oscillatory rates compared with present technology. Acoustic ventilation, the method of current invention, can decrease ventilation associated dysplastic changes in bronchial tree and alveolar scaffolding by delivering minute vibratory forces into end-bronchioles, and inflating alveolar sacs evenly, with less pressure. This new method of ventilation will have the lowest ventilation/perfusion mismatch compared to all other ventilation techniques. The machine described hereafter is to be interchangeably called acoustic ventilator (AV), acoustic ventilator machine (AVM), sonic ventilator (SV), or sound bloc ventilator (SBV). Very High Frequency Ventilator (VHFV) and Ultra High Frequency Ventilator (UHFV) can also be interchangeably used. In this document, wherever various parts of the machine are described, proximal means closer to the patient/subject and distal means farther away from the patient/subject.

The preferred embodiment pertains to a ventilator machine capable of generating pressure fluctuations of 0-60 cmH₂O at a rate of 0-60/min with added variable oscillations in the range of 1 Hz-50 KHz at intensities from 50-150 dB into a subject's airways via an ET tube, face mask, nasal prongs, laryngeal mask, and alike. These vibrations are well tolerated in the pulmonary system if the exerted sonic pressure oscillations remain under 150 dB which is the maximum set intensity of AV. Acoustic ventilator works mainly in pressures ranging from 3-15 cmH₂O and sound power pressures in the range of 45-110 which is considered quite safe and advantageous. We should remember that human voice can be as loud as 110 dB and obviously no one has ever been seriously hurt by yelling or singing loudly. Effects of loud sound on human/animal body has been studied [8][9].

Subjective tolerance of sound waves in the range of 50-100 Hz is about 150-155 dB according to some studies [2]. Another study showed emergence of painful internal body vibrations at 196 Hz with power of 160 dB which was continued for 5 min and caused no lasting ill effects on the subjects. At acoustic levels of above 190 dB, lungs begin to rupture [3].

Although not limited to these rates, acoustic ventilator functions mostly in sonic power range between 45-110 dB. Since sound power intensity is measured in logarithmic scale we should remember that sound intensity of 190 dB is 10,000 times stronger than the upper set limit of acoustic ventilator which is 150 dB. Also we should note that 150 dB is 10,000 times stronger than highest routinely used intensity of 110 dB which gives acoustic ventilators a truly wide window of safety. Bursts of intensities above 110 dB might be used randomly and briefly in acoustic ventilation, for example only for 0.1-10 seconds every 2-5 minutes, in select cases and particularly in the presence of severe pathology such as RDS or pulmonary edema. These high intensity sonic bursts, at times coupled with exertion of rapid decline in airway pressure (reverse hiccups), facilitate pulmonary excretion. Exact levels of required intensities and duration of the acoustic bursts remain to be studied.

Sonic ventilator synthesizes and blends pressure waves in frequency ranges below human voice (1-80 Hz), about human voice (80-500 Hz), above human voice (500 Hz-20 kHz), and ultrasound (20 kHz-2 MHz). Said bundle of vibrations will be exerted on a desired mixture of fresh gases with a variable airway pressure in the range of 0-60 cmH₂O fluctuating 0-60 times per minute. This will sustain satisfactory gas exchange with lower airway pressures and less FiO² compared with available technologies and thus, will have less ventilator induced lung injury (VILI). Acoustic ventilation maintains adequate oxygenation and CO² clearance in pressures ranging from 3-15 cmH₂O with 10-60 undulations per minute and sonic oscillations of 50-75 Hz plus a first harmonic in 150-235 Hz range, and a second harmonic, and so on (for example, oscillations of 65 Hz, 195 Hz, 325 Hz, 455 Hz, 585, and so on. In addition, harmonics (3, 5, 7, 9, . . . times of the original note) of each of these harmonics will be mixed and played together all at the same time. Non-harmonic/random tones, even single tones also work very well. It is very interesting that you can play your favorite music into an unconscious subject's lungs and keep them in good gas exchange status as long as the music is rhythmic and has certain variations or beats per minute. Interesting fact about music is that happy beats in any genre or culture are the ones with rhythms suitable for a respiratory acoustic boost, like panting or the music constantly played in our chest, the rhythm of heart. It could be one reason why so many people enjoy loud rhythmic music as it augments our breathing, beat by beat. Recent popularity of super strong kicker subwoofers in our cars and elsewhere might be another projection of our desire to enjoy this added free pulmonary boost with pounding of rhythmic music. If you stay close enough to loudspeakers of a rock concert your breathing will be facilitated by the speakers' vibrations. It would be interesting study to measure people's breath rates when they are in proximity of such powerful speakers. In one study, anesthetized dogs that were exposed to 127 dB sound intensity at 7 Hz stopped breathing as the strong vibrations in the air were taking care of gas exchange for them. When the infrasonic vibrations were stopped the dogs started breathing again. The dogs showed no ill effects after the study and were acting normally [2]. What happened in this study was a clear example of acoustic ventilatory assistance, the essence of current invention.

Although it might sound more like science fiction for now, but specially designed tones or tunes to deal with various pulmonary disorders through acoustic interventions will become a matter of exciting work and emergence of a new branch of medical sciences aptly to be called “Pulmosonics”, “Pulmorhythmics”, or “Pulmonary Cymatics”.

Since sound travels 343 meters per second in the air with normal temperature, pressure, and humidity (at 20° C. and sea level with humidity of 60%) then the wavelength of a 343 Hz tone should be approximately 1 meter, i.e. there is a node with minimal media (air) movement at 50 cm distance from the sound source (oscillator) and there is maximal air movement at 1 meter distance. Now, in acoustic ventilation, if we steadily go up on frequency the node will get closer to the source or the oscillator as the wavelength will be shorter. For example, if the frequency repeatedly goes up from 343 to 686 Hz then the node will move from the 50 cm distance to 25 cm and so on. This is the most efficient, fastest, and safest method of sweeping and cleaning up lungs particularly in life-threatening cases such as RDS and alike. Cymatics has shown this phenomenon very clearly in movements of sands on a plate which represents a 2D cymatics. Sonic cleaning/sweeping of lungs or “Pulmosonics” with acoustic ventilation/RBM would be cymatics in 3D.

On occasions, abrupt decreases in pressure or flashes of negative pressures of up to −10 cmH₂O can be employed briefly to help with removal of pulmonary secretions. This can easily be achieved by installation of a gas reservoir such as a tank/cylinder with negative pressure created by a piston or servo-motor. Said negative pressure reservoir is connected to patient circuits via a hollow tube and a valve that can open and allow air moving rapidly from patient circuit toward the reservoir creating “reverse hiccups”. The simple “reverse hiccup” contraption is not depicted in the figures of this invention. It can easily be controlled by a computer and proper software.

Sonic ventilation can also employ air-coupled ultrasound energy, particularly low-frequency ultrasound (20-100 KHz) which has a diverse set of industrial and medical applications. Low-frequency (LF) ultrasound is used in sonophoresis, ophthalmology, dentistry, histotripsy, thrombolysis, and extracorporeal lithotripsy. While new LF applications continue to develop, significant gaps in the coverage of safety standards for this frequency range still exist[4]

Acoustic ventilation employs ultrasound waves in the frequency ranges of 20 KHz-2 MHz. Breathable gases have a very high absorption rate on frequencies above 2 MHz and therefore, such frequencies do not have much direct use in acoustic ventilation except for heterodyning. Higher frequencies in the range of 2-10 MHz are not employed in sonic ventilation and its use will remain to be investigated[5]

Heterodyne waves form when two or more waves merge, for example when we combine al 200 Hz wave with a 1210 Hz, two new waves will be generated. One wave would be the sum of the two, in this case 2410 Hz, and the other wave would be their difference which is 10 Hz and that is the tremulousness we feel in the new combined tone. We use heterodyning in many areas in radio frequencies, tuning up our musical instruments, focusing or bending waves, etc. Heterodyning can be used in acoustic ventilation in different ways such as improving V/Q mismatch and facilitated pulmonary toilet by use of the tremulous waves and shifting nodes and wave lengths from smaller and deeper airways to more superficial and bigger airways. These effects of sound energy are well known in sound engineering/physics of sound and there is no need to resort to details in this disclosure statement. Heterodyning can also be used in ultrasound range to direct sound energy into the airways and potentiate effects in a fashion similar to what is currently used in “parametric sound emitters” better known as “long range acoustic device LRAD”[6]. Array of transducers, each vibrating at a certain frequency in order to focus the beam of sound, found in devices such as “Directed acoustic sound system” or “audio spotlight” by Holosonics[7], can also be employed in acoustic ventilation.

Currently, there are no ventilator machines capable of operating at these very high oscillation levels because they cannot pass pressure oscillations of this frequency ranges across a narrow tube such as an ET tube. Acoustic ventilation (AV) or SV/SBV comes with low operational noise level, precision control, and needed safety features along with added diagnostic capabilities. It can vibrate and influence different parts of lungs with specially designed harmonic and non-harmonic tones/tunes with variable frequencies, usually from lower to higher pitch like moaning sounds for example, in order to extract pulmonary secretions more efficiently.

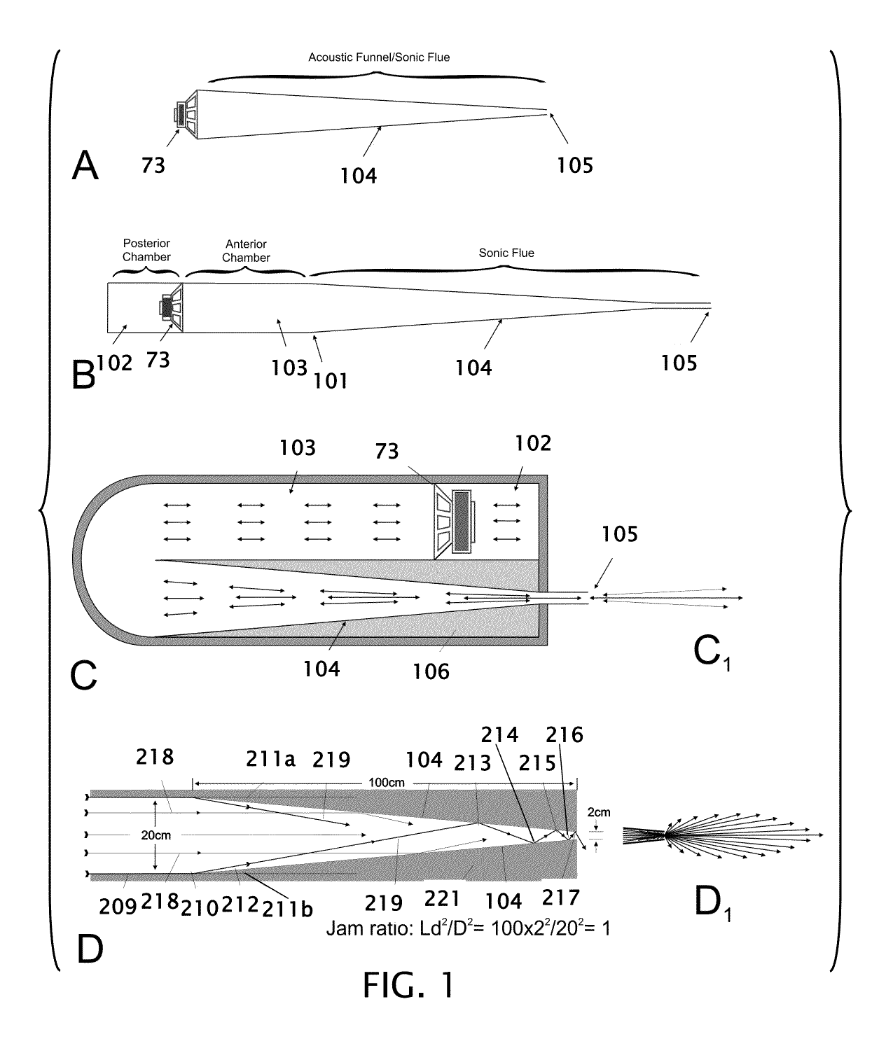

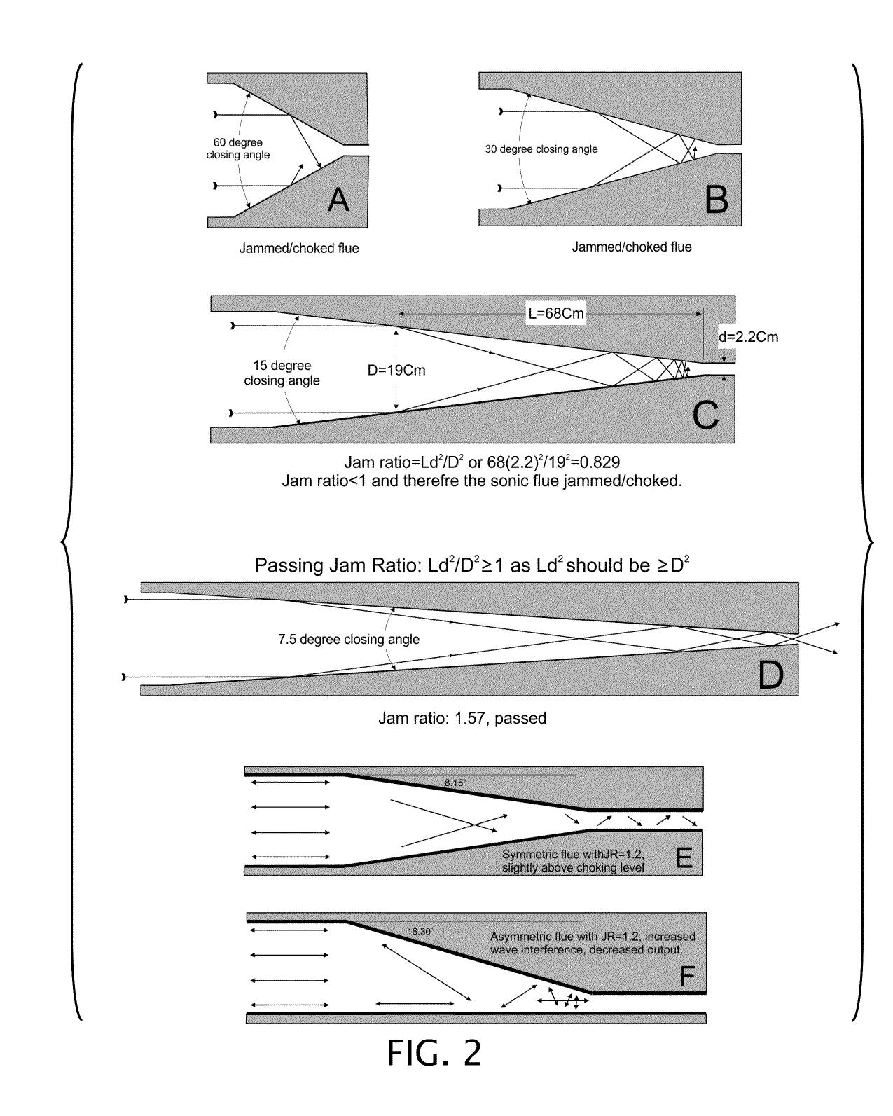

FIG. 1 depicts how sonic flues work by reflecting and directing sonic energy from a larger into a narrower tube such as an ET tube, without causing much acoustic wave interference and hence avoiding dampening of the oscillatory power and flow. According to simple laws of physics, the progressive, gradual and obtuse narrowing of the flue, as seen in FIG. 2, will cause the pressure to drop while the momentum of particle movement will increase as the funnel gets narrower and narrower. FIG. 2 shows how choking phenomenon can happen in sonic ventilation if gradual tapering of internal diameter of the funnel is acute and not obtuse. It also shows how sonic flue works and how to calculate the proper length of the waveguide in relation to its inlet and outlet diameters, in a simplified mathematical equation.

In the simplest assembly of the invention, an electronic signal generator or a computer, playing a variety of single tones or desired combination of tones, tunes, or even a rhythmically suitable music, can be connected to the input port of one or more electronic power amplifier(s) with its output ports connected to electro-acoustic transducers made of coil on magnet, coil on coil, magnet on coil, piezoelectric crystal, magnetorestriction, or similar technologies. As shown in FIG. 1A, said transducer is lodged at or near the larger or distal end 101 of a specially designed tube or waveguide similar to an overly elongated funnel 104 resembling a cone with open base and cut off tip 105. Said transducer 73 is installed in proximity of the larger opening of said waveguide with its diaphragm somewhat perpendicular to the long axis of the funnel and facing the narrow opening of the waveguide. As shown in FIG. 1A, said waveguide with a first larger-diameter opening and a second smaller-diameter opening has gradually narrowing lumen from the first to the second opening. Said progressively narrowing waveguide, called acoustic funnel or sonic flue, has gradually constricting walls 104 converging in such a mild angle as to guide sound waves forward, from the larger opening where the transducer is installed, toward the narrow end without significant dampening or acute reflections of the sound waves. This funnel is somewhat similar to the communication method in old navy ships or the way obstetricians used to listen to heart beats of a fetus before advent of Doppler ultrasound. The funnel has to have smooth reflective interior surface to avoid wave interference and energy loss, rough surfaces are not suitable. Glass, china, fiberglass, metal, veneered wooden boards, thick epoxy glass, PVC, ABS, and similar material can be used in construction of acoustic flues.

In reality sonic waveguides of acoustic ventilation are not that simple though. In FIG. 1B, you can see that the oscillator 73 is housed inside a box with the anterior chamber of the box 103 sealed onto the larger opening 101 of the funnel. The space behind the oscillator 102 is called posterior chamber which can be open to atmosphere or kept closed. Since such funnels are usually many feet long and will take a lot of space, and because sound waves do not lose much momentum travelling inside winding pipes, then as you can see in FIG. 1C in order to save space and make the machine more compact, the anterior chamber 103 leads into a bend and thus sonic vibrations made by oscillator 73 can be easily guided right into the sonic flue where according to simple laws of physics decreased diameter will decrease pressure and increase gas particle oscillation speed and swing. These effects have been shown by vectors inside the acoustic flue in FIG. 1C.

Referring to Bernoulli's Principle, Venturi Effect, Prandtl-Glauert Transformation, etc., it is well known in physics, that if you connect a conduit containing pressured gas (acoustic flue in this case) to a lower pressure system (patient airways) through a narrow pipe (ET tube) then gas molecules passing the narrow tube will have higher speed and lower pressure, allowing remarkable gas flow across even narrowest ET tubes. There are many equations for compressible flows moving at low Mach numbers (usually less than 0.3) and at higher Mach numbers which because of their vastness and complexity are not suitable for inclusion in this document. These equations are considered general knowledge of physics and accessible in myriad of ways (college and university libraries, internet, etc.).

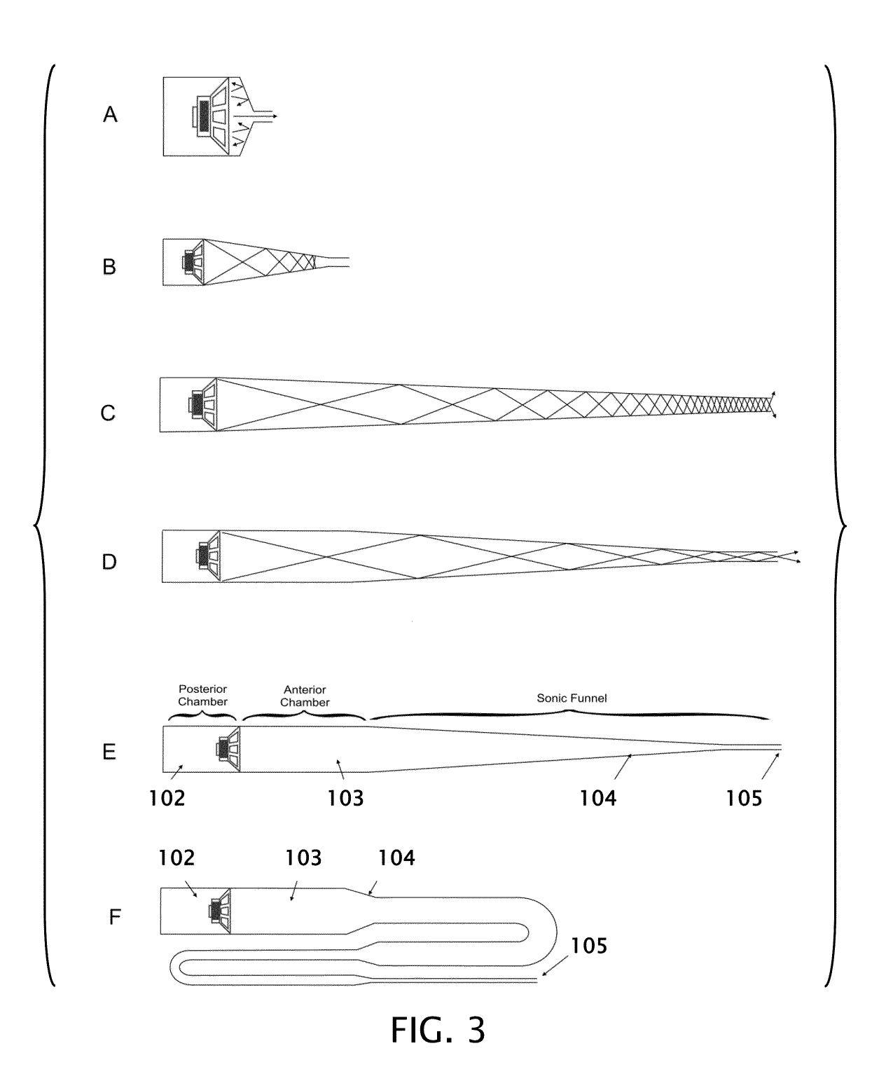

Although sound travels as longitudinal waves and light rays are transverse waves, but the way sound reflects off smooth surfaces is quite similar to visible light reflecting off a mirror, for example. A model of sound propagation behavior inside a gradually narrowing acoustic duct is shown in FIG. 1D in an overly simplified fashion. Using reflective properties of visible light, sketches of a working model of sonic flue were drawn on paper to mathematically comprehend why and how acoustic waveguides of current invention can move substantial flow across a narrow tube without resorting to significant pressure gradient. For simplification of calculations and as shown in FIG. 1B, amplified acoustic vibrations exiting parallel-wall anterior chamber 103 and entering funnel 104 hit the converging walls and just like light rays reflect off the smooth surface at the same angle they hit the surface because the law of reflection says that for specular reflection the angle at which the wave is incident on the surface equals the angle at which it is reflected. Based on this model, if they hit the reflective surface at a 30 degrees angle, then the reflected beam will be also make a 30 degrees with the surface. Although there are many other element such it is assumed that vibrations made by the oscillator have travelled inside a uniform and parallel wall tube 103 long enough (usually about 2-6 feet depending on frequencies used and diameter of the duct) that the waves entering the funnel are moving parallel to the long axis of the funnel. One of the widest reflections which will have the longest path inside the funnel has been selected and shown in bolder lines in FIG. 1D. This wave is farthest away from center of the funnel and will have the hardest time to get to outlet 105 as it will have the highest number of reflections before it can exit the funnel's narrow opening. Because the walls of said acoustic conduit are not parallel then the reflection angle of the “sound ray” off the inner surface of the funnel will increase after each reflection according to the degree of convergence of the walls. If the walls converge sharply, like in a short and shallow funnel, then most of the sound rays will be reflected back to the oscillator (FIGS. 3A and 3B) but if the walls merge gradually, like in a long and deep funnel (FIGS. 3C and 3D) then sonic vibrations can be focused into the funnel's outlet and exit. Obviously, short shallow funnels have obtuse closing angles and long deep funnels have acute closing angles. Closing angles (FIG. 3) form if we let the walls of the funnel intercept. If the closing angle of an acoustic funnel is acute or small enough, 3-5 degrees for example, then the sound rays will exit the outlet after one or more reflections depending on their proximity to the longitudinal axis of the funnel. Obviously, sound rays closer to the long axis of sonic flue will pass through the outlet hole intact without any reflections. Apparently more obtuse closing angles, 30-180 degrees for example, will become more and more opaque to sound waves.

If the farthest from center rays pass through the waveguide's outlet hole the acoustic flue/funnel is considered sonically conducive, otherwise, it will be a sonically unconducive (choked) flue, refer to FIGS. 2&3. Length of the funnel, diameters of its inlet (oscillator) and outlet (size of the ET tube) are important parameters predicting an acoustic flue's conduciveness (open to sound waves or not). Size of the ET tube, diameter of the oscillator(s), and subsequently length of a sonic flue are all predicted by age/size of the patient. Sonic vibrations travelling down non-compliant tubes do not dissipate quickly and thus, long acoustic waveguides are most conducive.

As shown in FIG. 1D, the convergence or closing angle (CA) of any symmetric sonic flue is equal to the sum of angles 211a₁ and 211a₂. Guide angle (GA) 210 which will eventually determine the chances of a sound beam to exit a funnel or not can be calculated as 180 minus the CA. In FIG. 1D, closing angle (α) is 10 degrees, therefore, the first GA 210, called δ₀, should be 180°−10°=170°, the second GA 213, called 81, will be 180°−2×15°=1500, and for GA angle 214 or δ₂=180−2×25=130° and angle 215 would be δ₃=180−2×35=1100 and so on. The easy way to calculate guide angle δ is: δₙ=180−(2nα+α) where α is the closing angle and n is the number of reflections after the first one. Obviously, after few reflections, δ will be getting closer to zero, and apparently zero delta means a beam reflected back onto itself, or jammed. It means that the wave will not exit the funnel and will choke the flue as it jams inside the funnel and creates destructive interference with other waves.

Multiple linear/simple sonic flue models were used to analyze relations between the length of the waveguide with size of the oscillator and size of the ET tube to be used and therefore, “jam's Ratio” (Ld²/D²) was figured to anticipate if a funnel is sonically conducive or not. In jam's ratio, L is the length of the acoustic flue, d is the diameter of the outlet (ET tube size), and D is the diameter of the inlet (or the oscillator). If jam's ratio is greater than one then the flue is sonically conducive, and if jam's ratio is less than one, then the flue in sonically opaque or unconducive.

The ratio will also help us estimate the required length of the sonic flue. In this fashion length of a suitable acoustic flue can be calculated as:

L≧D2/d², for example if the oscillator is 20 Cm wide and ET tube is 2 Cm wide then 400/4=100 and that means the length of the flue should be at least 100 Cm long. As mentioned before, when jam's ratio is more than 1 the acoustic flue is considered open or conducive. Acoustic flues with jam Ratio of less than one are not desirable in construction of acoustic ventilators although such flues may still work but they tend to waste energy, create noise pollution, be less predictable, and have attenuated energy output. FIG. 2 compares various conducive (D&E) flues with some unconducive ones (A, B, C, and F).

Oscillator diameter, closing angle, length of the sonic flue, frequencies and intensities needed, all depend on the size of the subject to be ventilated and the medical condition. Although closing angle can be as high as 30 degrees or even more, depending on size of the funnel's inlet and outlet, but in practice efficient sonic flues have much smaller closing angles usually around 3-10 degrees. In one in-vitro test, a 12 Cm diameter oscillator generated 15 L/min flow at the tip of a 6.0 ET tube with 45 W continuous power output at 65 Hz with a sonic flue 3 meters long. This test proved that acoustic flues with jam ratio of less than one still work, albeit in a suboptimal fashion as the oscillator was quite noisy and had attenuated output. In one type of acoustic flue (FIG. 3F), sequential short funnels are interconnected by parallel-wall tubes. These sonic flues are called serial acoustic cascades. If designed properly, they work as good as a regular or maze type of acoustic flues. Helical flues are considered superior.

Size of the oscillator's diaphragm should be selected according to needed frequency range which is dictated by size of the subject or subject's lungs. For example, newborn lungs can probably be satisfactorily ventilated by a 5-7 Cm, Infants by a 7-10 Cm, children by a 10-15 Cm, and adults by a 15-20 Cm in diameter oscillator in an acoustic ventilator assembly. These numbers are rough estimation because human subject studies have not been done yet. As a general rule, the larger the size of the lungs to be ventilated the wider the diameter of the oscillator, and vice versa.

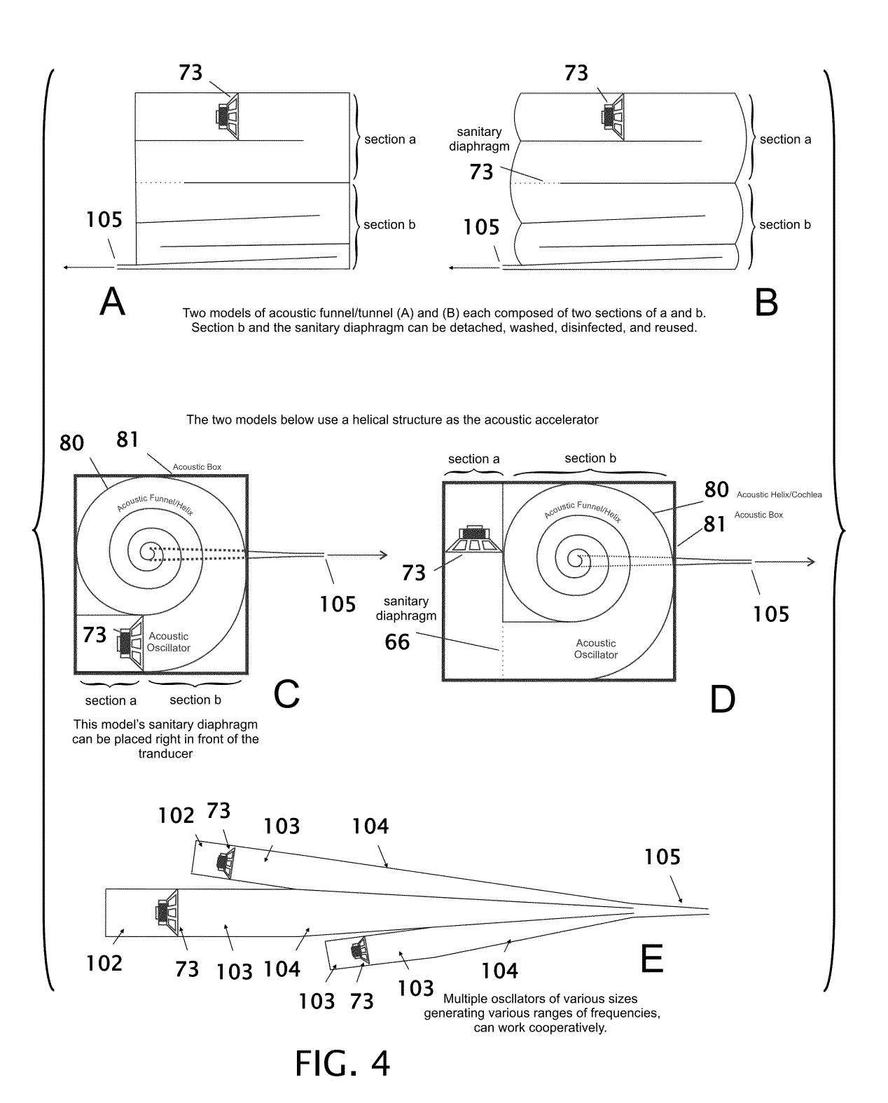

In one embodiment of the invention, multiple sonic flues conjoin to form one unit. Each of the conjoining flues has a different size oscillator and is capable of playing a different range of frequencies. A simple linear form of conjoining acoustic flues is shown in FIG. 4E. Obviously, the flues can be of any type or shape already discussed, for example, helical, maze, cascade, or various cross sectional, and so on. Normally, smaller and shorter flues have smaller oscillators and play higher notes/tones/tunes, and bigger ones play the lower frequencies. In this setting, one or more of the oscillators can operate in ultrasonic range, usually the smallest, and conversely, one or more of the conjoining oscillators can operate in infrasonic range, usually the longer and wider ones.

Although sound propagation in ducts and waveguides have been amply studied but unfortunately literature regarding properties of sound propagation down a progressively narrowing tube is scarce [11], [12]. Sound propagation equations in ducts, with consideration of gas temperature/humidity, sound intensity, pressure changes, etc. are complex and extensive [10], and will not be discussed here.

Length of the sonic flue is important to be measured appropriately as well. One simple and easy way of finding the approximate right length is using jam's equation (L≧D2/d2), as discussed earlier. FIG. 3A shows a very short funnel with closing angle of almost 150 degrees which obviously is way off the chart in setting of acoustic flues (typically with closing angles below 10 degrees). FIG. 3B shows a waveguide which is still not long enough and as seen, acoustic waves have jammed inside the funnel and choked the flow. FIG. 3C shows a long but less desirable flue with low output/flow and FIG. 3D shows how addition of a length of parallel wall tube can improve output/flow. FIG. 3E shows names of various parts of a typical simple linear sonic flue with posterior chamber 102 and anterior chamber 103 both with non-converging walls. Said chambers create strong sonic resonance and introduce them to funnel 104 where most of the sonic energy is focused onto outlet 105. FIG. 3F, shows a special model of a sonic flue with multiple short funnels and interconnecting parallel-wall tubes in between. These are examples of tubes with round cross section while any geometric shape for cross section of such tubes can be functional and acceptable.

Other functional models of the flue are shown in FIG. 4 where a sanitary diaphragm 66 is also depicted. This diaphragm can be placed in various spots in the course of the funnel/flue. As you can see in the first 2 top varieties (4A &4B) the closing angle of the flue is not symmetrical and although symmetrical flues are more desirable but in practice asymmetrical flues work almost as good as symmetrical ones. Asymmetric flue designs have the advantage of easy manufacturing and cheaper production. FIGS. 4C and 4D depict a special design of more efficient acoustic flue, named acoustic accelerator where similar to our ears' cochlea a spiral or helical formation is intended to guide sonic energy efficiently into smaller tubes fit to be connected to an ET tube. These particular types of acoustic flue showed about 20% improved output/flow in the lower frequency ranges around 30-65. The enhancement in flow was less profound in higher frequencies. These helical or spiral sonic flues, or sonic accelerators, can be manufactured in two matching halves which can be easily separated, washed, disinfected, and used again. Porcelain types, although expensive, work the best. Sanitary diaphragm can be used right on top of the oscillator, similar to Carefusion's 3100 HFOV, or farther down the acoustic ducts.

Actual gas exchange, i.e. introduction of fresh inspiratory gases and removal of high CO₂ content expiratory gases, can take place at one or more areas inside sonic flue, inside a chamber called air-hub, or inside patient's trachea. Obviously, introduction of fresh inspiratory gases and removal of expiratory gases do not have to happen topographically at same spot on any part of the machine. For example and as it will be explained later, fresh gases can be introduce into the flue, air-hub, or directly into patient's airways while expiratory gases can be expelled from patient's airways, air-hub, or any part of the flue. In order to accommodate all possible functional configurations of actual gas exchange in the machine, few different forms of sonic flue was designed.

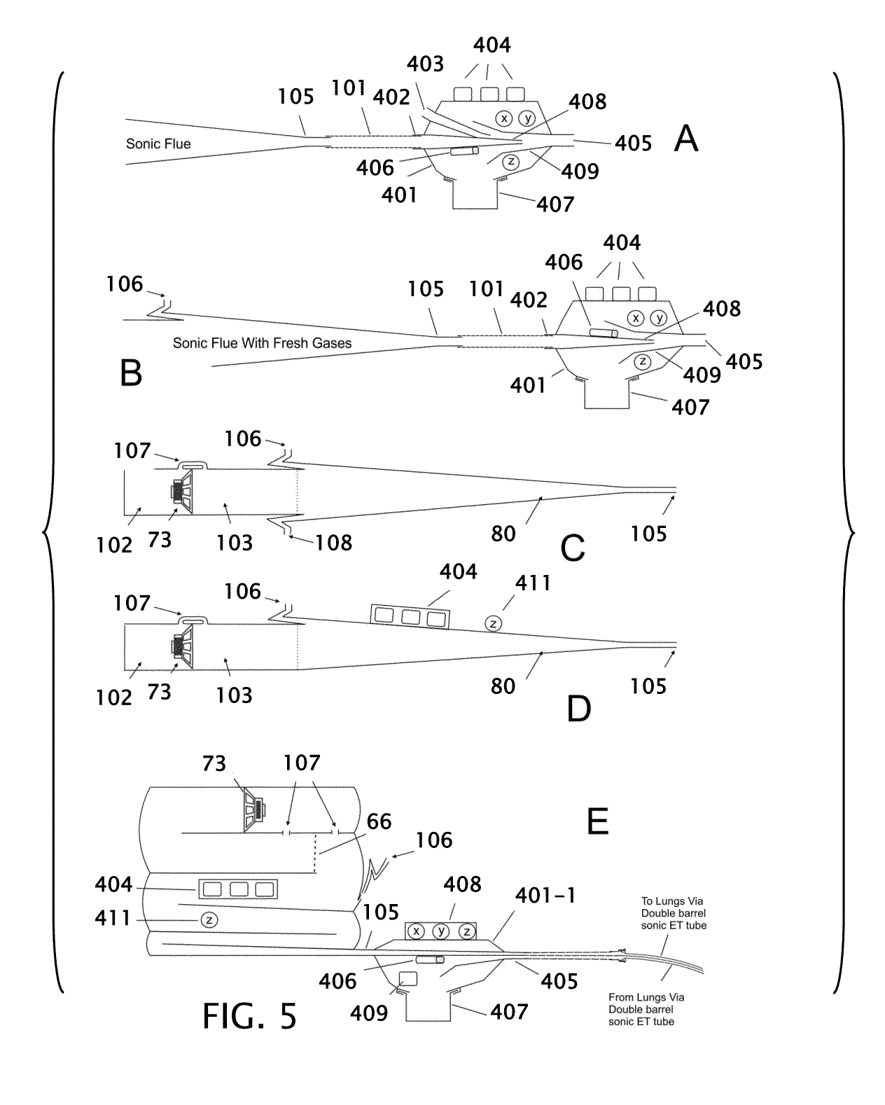

As shown in FIG. 5C, the simplest form of a sonic flue, fresh gases can enter a sonic flue via port 106, and mixed gases can exit the flue through port 108 where a simple mechanical pressure limit valve predicts the outflow of gases through 105 into patient's airways via an ET tube, facial mask, nasal prongs, laryngeal mask, or similar devices. These ports can be located at any point along the length of the waveguide.

FIG. 5D on the other hand shows a more complicated form of a linear acoustic flue. It shows how fresh gases can enter a sonic flue via port 106 which can be located at any point along the length of the flue. Said fresh gases, energized with sonic vibrations, will subsequently pass through sonic outlet 105 and into patient airways via an ET tube/facial mask/laryngeal mask/nasal prongs, etc. Apparently, this will ascertain needed positive gas pressure inside the flue which can fluctuate 0-60 times per minute between 3 to 35 cm H₂O pressure, for example. This pressure will be sustained, balanced and controlled by adjusted flow of heated and humidified mixture of gases in desired proportions entering the flue via port 106 and exiting through sonic outlet 105 (to the patient's airways) or through control/dump/limit valves 408 released into atmosphere (at times through an optional sanitary filter).

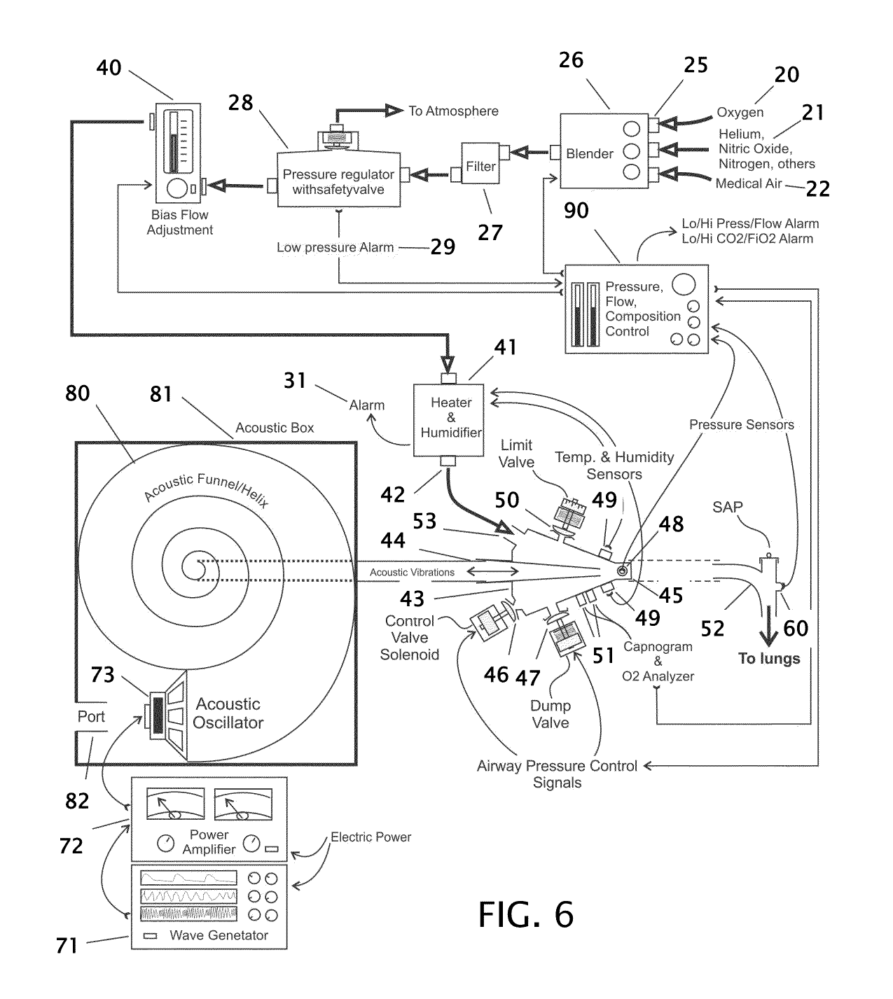

In most ventilator machines, control valve is an inflatable sac of air that blocks a spout connecting expiratory gases to atmosphere. The pressure inside the sac will predict patient circuit and eventually airway pressures. In current invention this valve and the dump valve which opens only when pressure reaches a certain point are both made of electronic elements as shown in FIG. 6. They work with a solenoid valve blocking said expiratory gas opening or spout to the atmosphere. Default setting of control valve is “close” i.e. if anything went wrong with the machine it stays closed so pressure in patient airways would not be lost. In this case, pressure will go higher and higher till dump valve will open and let the air out and keep pressure at a certain set level. If control valve and dump valve both were malfunctioning then limit valve will not allow the pressure go above a certain level. Limit valve is mechanical and totally independent of the machine's operations. It always is set manually by the personnel. Most of the time and under routine conditions and in a well-built ventilation system only control valve is constantly changing tone and maintaining airway pressures, and normally dump valve or limit valve rarely if ever should be needed to intervene. In AVM the tone of control valve or it's pressure on the spout is dependent on the electric current in the solenoid and the magnetic field generated by such current that keeps the iron rod located in the center of the coil in proper position. The iron rod is connected to a smooth rubbery cushion which can block air exit from patient circuits through the spout. These electric valves are controlled electronically by AVMs control panel which will be discussed later. All such technologies already exist and there is no need to delineate such mechanism here any further.

For simplification, instruments such as capnogram, O₂ anlyzer, pressure, temperature, humidity sensors (designated as 51, 48, 49 respectively in FIG. 6) are collectively marked as 404 in FIGS. 5A, 5B, 5D, and 5E. Control, dump, limit valves (designated as 46, 47, 50 respectively in FIG. 6) are collectively marked as 408 and shown as circles labeled x, y, and z in FIGS. 5A, 5B, 5D, and 5E. Although function and role of these sensors and valves are well known in the art but they will still be discussed further later in this document.

Special attention should be given to installation of any ports, sensors, or devices around sonic flue to reduce operational noise of acoustic ventilator machine (AVM), prevent acoustic energy ooze, and circumvent destructive sonic wave interference inside the flue. For example and as seen in FIGS. 5D and 5E, port 106 can be designed with angulations and slanted entry point. Contact surface of sensors or valves with sonic flue should be flushed with interior smooth surface of the flue and not intrude into its space and create acoustic disturbance.

Another feature of some acoustic flues/accelerators is a low-pass tube 107 connecting anterior chamber 102 to posterior chamber 103 in order to equilibrate pressure on either side of the oscillator as to avoid malfunctioning of the oscillator secondary to such pressure gradients between the two chambers. This tube is usually very narrow and can have a sanitary filter inside its lumen which can be changed periodically. As a diagnostic tool and in certain cases, the filter can be sent to lab for bacterial/viral studies/cultures.

In one embodiment of the invention depicted in FIGS. 5A & 5B, the proximal end of sonic flue 105 is connected to a pressure control chamber called acoustic air-hub or simply air-hub where fresh gases blend with patient exhaust gases. As seen in FIG. 5A, heated humidified mixture of blended gases with desired flow enter the hub through port 403 and get energized with sonic, infrasonic, or ultrasonic vibrations that exit sonic flue outlet 105 directly or after said sonic vibrations pass a distance inside tube 101 connecting the main body of AVM to the bedside air-hub. Said vibrations entering port 402 into the air-hub's own little sonic flue 408 where a funnel or similar shaped receptacle 409 mixes said mixed fresh gases with said vibrations and sends/diffuses them down tube 405 which will eventually be connected to an ET tube for example. Obviously mixed inspiratory and expiatory gases inside the hub need to exit the hub in order to make room for gases to enter the hub via port 403. For this purpose and also in order to maintain pressure inside the hub, a control valve is adjusting pressure by letting some of the gas out into atmosphere. Mechanism of action of this valve was briefly described earlier. These valves and technologies involved in their control already exist. For safety reasons, this valve is accompanied by 2 other valves called dump valve and limit valve. These three valves are lableled as x,y, and z in FIG. 5A and will be discussed further later in the document. At times, only one or two valves can be designed as such in order to assume duty of all three valves but most of the time it is preferred to have at least 2 valves, for safety purposes.

In some embodiments of the invention an ultrasonic transducer is located inside the air-hub in proximity of the funnel 409 of port 405. Ultrasound is used to cast ultrasonic rays into the airways from a closer range compared to ultrasound beams coming from the sonic flue, to improve gas exchange and expectoration. Said air-hub transducer(s) at times is/are used as a diagnostic tool as well in order to read back ultrasonic reflections from patient airways and detect consolidation, mucus plugging, or changes in compliance.

In some models of the air-hub and as seen in FIG. 5B, inlet 402 is connected to sonic outlet 105 of sonic flue of the type seen in FIG. 5D via a regular respiratory tube or preferably via a non-compliant but flexible tube 101. Port 402 will progressively become narrower in a fashion similar to a smaller size sonic flue 408 where pressured vibrating gases coming from outlet 105 of sonic flue via tube 101 and port 402 will make Ventury effect at the narrow proximal tip of air-hub flue 408. Proximal portion of air-hub outlet 405 forms the patient circuit and takes the vibratory fresh gases to patient's airways. Air-hub's flue can be linear, helical, or any other type discussed in this document. As shown in FIG. 5B, said port 405 makes a funnel shaped structure inside the hub 409 where spurts of vibratory fresh gases (similar to what is shown in FIGS. 1C₁ and 1D₁) will be received and transferred down to the more proximal portions of tube 405 and eventually to patient's airways via an endotracheal tube, a laryngeal mask, a face mask, or alike. Said air-hub encased in casing 401 is composed of pressure, temperature, and humidity sensors 404, patient circuit 405, sputum collection tray 407, ultrasound emitter 406, acoustic vibration port 105, and fresh gas inlet 403 (5A only). In FIG. 5B, the air-hub does not have the inlet 403 for fresh gases. Air-hub shown in 5B works with a specially designed sonic flue shown in FIG. 5D. In this case, fresh gases first enter the flue via port 106 and then enter the hub via sonic outlet 105 which becomes sonic inlet 402 of the air-hub. This model is more efficient as fresh gases penetrate farther down the airways. Tray 407 need to be removed, washed, and disinfected periodically as it fills up with sputum, blood, or other secretions from the lungs. Disposable trays or disposable liners for the trays can be used as well. Said tray can assume many different shapes and sizes. It might even be hooked to suction means in some models.

Since air-hubs usually have positive gas pressure inside their chamber, then each unit should have at least one extra or spare sputum tray to immediately replace the one which needs to be cleaned so the pressure inside the chamber will be restored promptly, and prolonged collapse of lungs can be avoided consequently. In one model, a drawer form of the tray can be designed to avoid such airway pressure drops although acoustic ventilator will have no problem re-inflating collapsed lungs with its sonic vibrations safely and rapidly. Control/dump/limit valves which will be discussed later are shown in FIGS. 5A and 5B as circles labeled x, y, and z. Location of placement of these valves on the air-hub may vary. Air-hub model shown in 5B works well with sonic flue shown in FIG. 5D. Air-hubs can carry a capnogram and O₂ analyzer which are not shown in the figures.

As shown in FIG. 5C, another way of mixing gases is to use a special type of sonic flue with inlet 106 for introduction of fresh gases into the flue and outlet 108 connecting the interior space of the flue to outside environment/atmosphere for removal of mixed exhaust gases. Evidently, such inlet/outlets can be installed at any point down the length of any type of the acoustic flues we have discussed in this document. The angle of introduction of such connections (106, 107) into the flue(s) is important in order to avoid spoiling sonic energy, and prevent noise pollution. In yet another type of sonic flue, there is only on gas inlet 106 as shown in FIG. 5D. In this latter type, fresh gases enter through inlet 106 and exit through sonic outlet 105 of the flue; i.e. the sonic outlet 105 will act as the gas outlet of the flue, where outlet 105 can be connected to a narrow leaking ET tube so the expiratory gases do not enter the flue and instead exit to the atmosphere from patient's airways passing by the leaking points around a narrow ET tube. Such ET tube adjustment and placement is known in art of ventilation. In this particular setting of acoustic ventilation it is preferred to select an ET tube with a cross sectional area less than half of cross sectional area of patient's trachea, and at times even less than a quarter because the tube might block expiration at tighter sections of airways such as vocal cords, glottis, or epiglottis. Some ventilation technologies already use leaking ET tube mechanism. This method might be preferred when ventilation is anticipated to be for a short period such as minutes to hours.

In yet another embodiment, shown in FIG. 5E, which is suitable for longer ventilation needs and is one of the safest and most efficient types of acoustic ventilation assemblies, sonic flue's outlet 105 connects directly to the first lumen of a double barrel ET tube where the second barrel or lumen acts as an exhaust duct and is connected to air-hub or a similar device where an adjustable safe level of positive airway pressure can be maintained and monitored. In this case, heated humidified desirable gases enter the flue directly instead of entering the air-hub. In this setting, the air-hub can be equipped to a capnogram 409 while pressure, temperature and humidity sensors 404 are installed directly on the walls of the sonic flue. Control/dump/limit valves installed on the walls of the flue are indicated as x, y, and z in FIG. 5E.

As mentioned earlier, an electro-acoustic or sonic transducer is sealed onto the larger or distal opening of a progressively narrowing tube or waveguide resembling an excessively elongated funnel in the fashion showed in FIGS. 1, 2, 3, 4, and 5. Said symmetric/asymmetric waveguide gradually narrowing from first distal end to second proximal end can have a round, oval, square, rectangular, or any other geometric shape cross section. Combination of various cross sectional geometric shapes will be operational as well. The proximal or narrow end of said waveguide can be attached to flexible but non-compliant tubes which deliver the oscillations to the ET tube or ET tube adapter. This happens directly in some models of AVM, and in some other models after passing through a gas exchange chamber. Although sonic flues can be linear or straight but since sound waves do not get significantly distorted/dampened inside a bending waveguide, and in order to save space and improve portability, said sonic flue/accelerator can be built in a helical or labyrinth like shape.

Although sound waves are longitudinal waves and differ from light waves which propagate as transverse waves but similar to light, sound waves reflect off smooth surfaces in symmetrical angles like reflection of light in a mirror. Therefore, the angle of reflections and manner of propagation of sound waves inside a progressively narrowing duct or waveguide can be figured out using a light reflection model. This model is useful in designing an acoustic flue with minimum destructive wave interference and maximum constructive wave interference toward the tapering proximal opening of the waveguide. According to FIGS. 2A, 2B, and 2C closing angle of a sonic flue should be less than 15 degrees to direct reflections of the generated acoustic waves in a one directional course toward the narrow end of the said sonic flue and eventually into the ET Tube with least number of acute reflections and destructive interference. Waveguides with slightly larger closing angles will still work but the energy waist and machine noise will be higher. Closing angles are preferred to be symmetrical relative to their longitudinal axis or path of wave propagation as asymmetrical angles are seemingly less efficient. The sides or walls of the sonic flue should continue to slowly converge till the end of the waveguide where the funnel's proximal end approximately matches the lumen of the ET-Tube or less, although slight deviations from this continuum do not cause the machine to stop working but then again there will be some sonic energy interference and loss. In some models of sonic flue, the walls of the waveguide remain parallel for a length of few feet before the walls start to converge. This will allow the vibrations build up strong harmonic tides before entering the funnel and therefore, overall output of sonic flue will be improved.

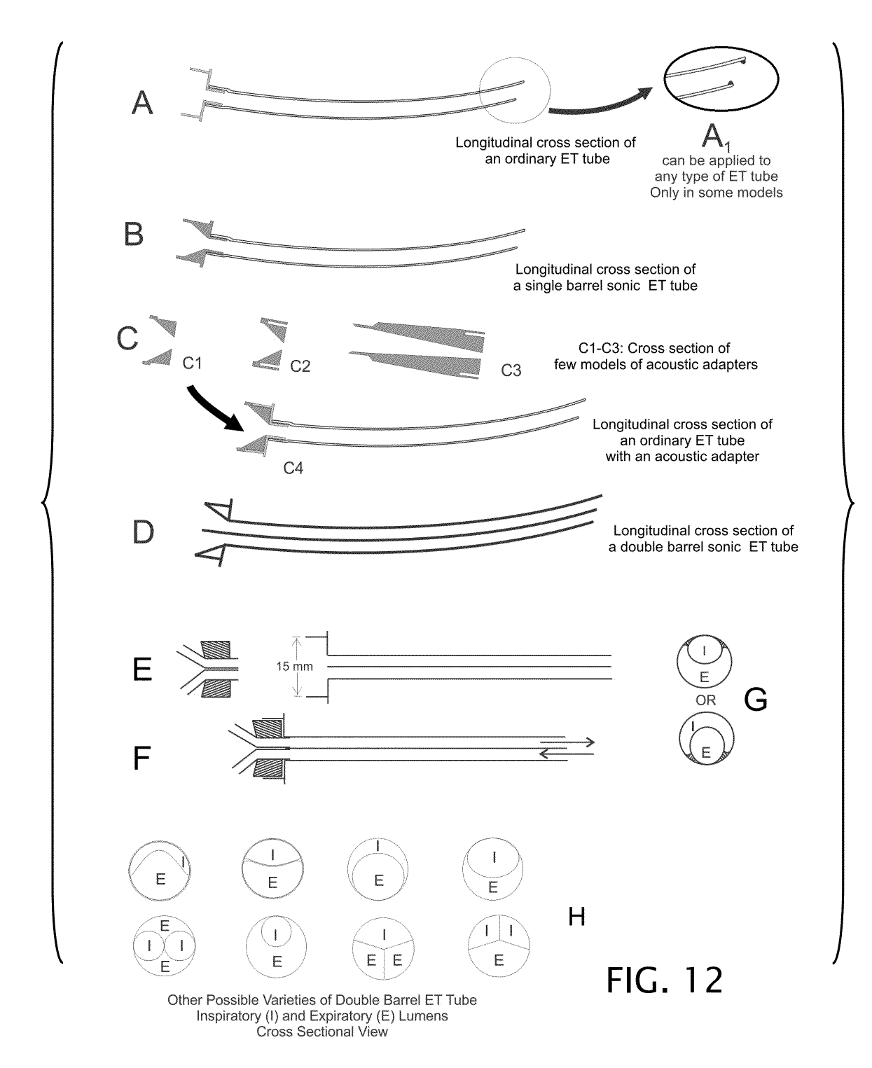

In order to fit the smaller or proximal outlet 105 of sonic flue to ordinary ET tubes of various sizes, or turn regular ET tubes to acoustically friendly ones, there are different size adapters which have progressively narrowing lumen similar to a small sonic flue. These adapters will prevent acute deflections of acoustic waves and avoid destructive wave interferences, energy dissipation, and noise pollution. Not limited to these few forms, different models of acoustic ET tube adapters are shown in FIG. 12C (C1, C2, and C3). In section C4 of the figure, proper location of the adapter on a regular ET tube has been shown. Cross sections of an ordinary ET tube (FIG. 12A), an acoustic ET tube (FIG. 12B) and an ordinary ET tube with an acoustic adapter inserted (FIG. 12C₁) are shown as well.

As you can see from FIG. 3, where sonic flue is compared with current technology, the closing angle in acoustic ventilator is usually around 5-10 degrees while the closing angle in some models of HFOVs, Carefusion's 3100A/B for example, is a whopping 150 degrees which as you can see in FIG. 3A, will create significant wave interference and energy loss. That is one of the main reasons these machines are pressure dependent, energy inefficient, noisy, imprecise, and limited to certain frequency range. A closing angle of 5-10 degrees will give rise to a length of sonic flue of few feet to few yards, depending on the size of the subject to be ventilated which dictates size of ET tube and size of the oscillator. In order to make sonic flues more compact, the waveguide can be curved and twisted in the fashion shown in FIGS. 3F and 4A, 4B, 4C, and 4D. Folding or bending of these tubes will not cause much destructive wave interference or energy loss. It is important to use non-compliant non-vibrating material in the make of the waveguide with smooth and non-absorbent inner lining. Obviously, sonic flue made of compliant or flimsy material with rough or sound absorbing inner lining would not be ideal. Sonic flue can be made of flexible material but it has to be non-compliant. In prototypes, thicker glass or epoxy glass (>3 mm in thickness), fiberglass, veneered wood, hard plastic (such as PVC, ABS, or similar), and glazed pottery were successfully used.

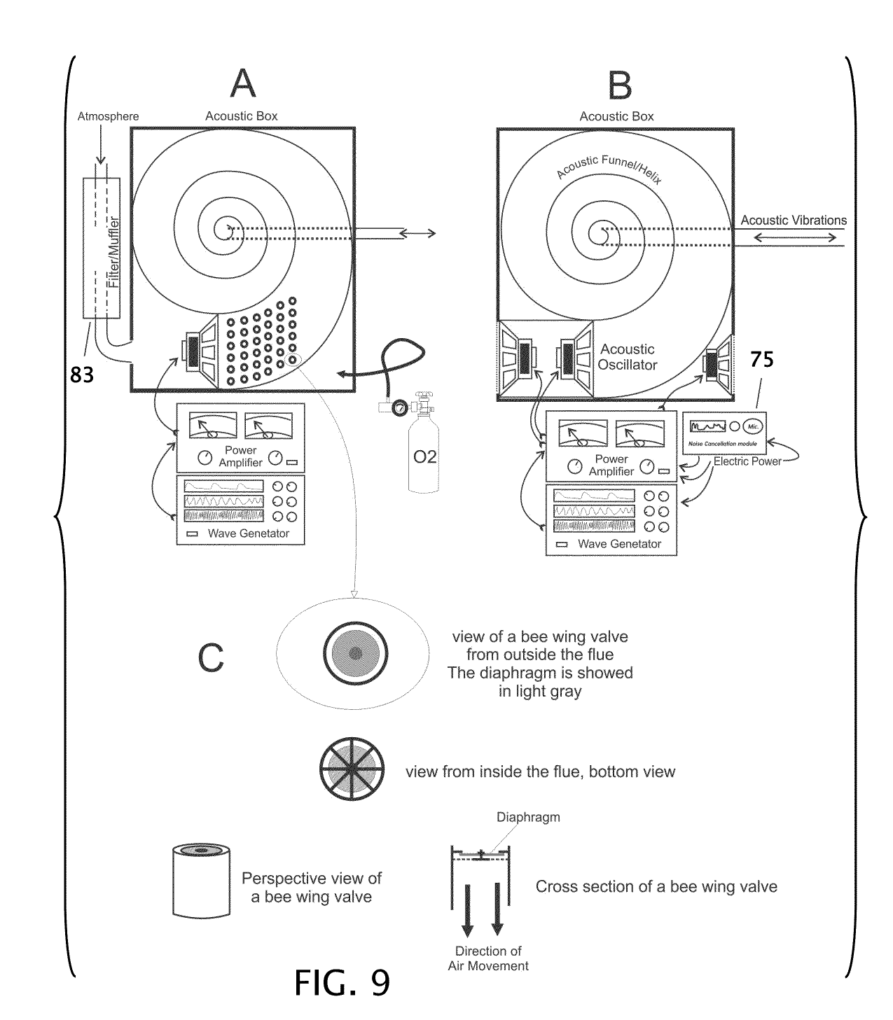

As mentioned earlier, in some sonic flue models in order to potentiate sonic vibrations and reduce sonic interference, the oscillator is mounted in the parallel-wall portion of the waveguide as shown in FIGS. 3 and 4. This will allow enough space in the anterior 103 and posterior 102 chambers of the oscillator to create much needed resonance, just like a loudspeaker box potentiating the sound intensity. The posterior chamber may vary in size, shape, and dimensions. It can be closed, and in cases, interiorly lined with sound absorbing material such as foam or alike. Said posterior chamber 102 can also have a port just like a loudspeaker port which can remain open or connected to a muffler 83, as seen in FIG. 9. Anterior chamber of the oscillator can be few feet long, with parallel walls and any geometric cross sectional shape, before its open end gets connected and sealed onto the constricting or narrowing section of the sonic flue. When the waves move toward the narrow or proximal end of the flue/accelerator they will be squeezed together in a narrower column of air, allowing them to pass through even narrowest ET-tubes. Sound energy travelling in this fashion will be able to move air and diffuse gases down to the smallest airways, a marvelous advantage of acoustic ventilation, absent in currently available technologies. In some embodiments, anterior and posterior chambers are completely symmetrical, each entering a sonic flue of their own and obviously, the waves exiting the two sonic flue outlets indeed are anti-phase compared to each other. Anti-phase waves eventually enter patient airways directly connected to an ET tube for example, or through the air hub and at times via a double barrel ET tube which is shown in FIG. 12 and will be explained later.

Generated pack or bloc of vibrations after passing through the air-hub can pass through non-compliant flexible tubes to deliver the agitated gas into a subject's airways via an endotracheal ET tube, a mask, or simply and directly right into a box/cabin/room where the subject is positioned. Since AV is capable of sufficiently ventilating alveoli with employment of lower airway pressures then a noticeable improvement in pulmonary and systemic blood circulation will be achieved compared to other methods of ventilation. This ability of AV becomes crucially important in conditions where patients are at the verge of cardiopulmonary compromise such as cardiac failure, shock, premature newborns, pulmonary hypertension, and very ill or very old patients. Initial in-vitro studies with sonic flue and test lungs hooked on a Fleisch pneumotachometer in February of 2012 showed a significant ventilatory power of more than 15 L/min (unpublished data). In particular, frequencies of 30 Hz-200 Hz were very effective in actively ventilating test lungs. In another prototype of the machine frequencies of 50-75 Hz was the most effective in generating a strong flow of gas through the tube system. Subsequent in-vivo studies in the same year proved powerful ventilatory command of acoustic oscillators in a rat model (unpublished data). Results of the above mentioned studies were formally presented by the inventor at the International Conference of High Frequency Ventilation in Utah, USA in April of 2013.

### DESCRIPTION OF PREFERRED EMBODIMENTS

Acoustic ventilation in its simplest form is schematically represented in FIG. 6. Medical air 22, or filtered room air from any type of medical grade air pump, and oxygen 20 with/without other breathable gases 21 including but not limited to Helium, Nitric Oxide, and Nitrogen, typically pressurized in the range of 30-90 psi, enter a gas blender 26 through designated inlet ports 25.

The mixtures of gases pass through a medical grade filter 27 and enter a pressure regulator 28 which brings down the pressure of gases from 60-75 psi, for example, to 1.4 psi, approximately 0.1 bar, or around 100 cm H₂O. Such pressure regulators that can be used are known in the art and already available in the market. In some embodiments, there is an alarm system 29 at this level which indicates low pressure of gas in tubes coming from the source through the filter 27.

The blended gases in desired proportion, usually expressed in fraction of inspired oxygen or FiO² (21%-100%) go through a flow adjustment device 40 or a flow meter. On average, a flow of up to 40 L/min is sufficient to ventilate even largest human lungs but there are no limits to this as acoustic ventilators can also be used in veterinary medicine and in very large animals such as elephants where higher flow rates might be needed. The blended and metered flow of gas then will pass through a heater & humidifier 41. Such heater & humidifiers that can be incorporated in the machine are well known in the art and are already available in the market.

The desired flow of heated and humidified gases 42, usually expressed as liters per minute (L/min), enter the sonic air hub 43. The sonic air hub 43 is a pressurized gas chamber proximal to patient circuits and adjacent to means of connection to airways such as an ET-tube. The pressure in this chamber closely resembles patient airway pressures because sonic vibrations equilibrate pressure on either side of the ET tube without resorting to very high pressures like all HOFVs. In order to sonically energize fresh gases that enter air-hub 43 via inlet 53, an inlet 44 is designed to allow sonic oscillations enter the air-hub in the fashion shown in FIG. 6. The waveguide introducing acoustic vibrations into the air-hub forms a Venturi effect and not only pushes mixed gases down the outlet 45 and patient airways but also pulls the mixed expiratory gases back into the hub where fresh and old gases constantly mix and eventually exit the hub via control valve, and occasionally through dump or limit valves. Sonic air-hub 43 in some embodiments also has a mechanical safety valve 50, a solenoid control valve 46, a solenoid dump valve 47, pressure sensors 48, temperature & humidity sensors 49, O2 analyzer and capnogram 51. As shown in FIG. 5B, the hub 43 can also have one or more ultrasonic transducer 406 adjacent to patient circuit in some embodiments which can direct a beam of vibrations of 20 KHz-2 MHz into the ET-tube.

In a preferred embodiment of acoustic ventilation a sonic flue of any form such as linear, helical, maze or labyrinth receives fresh gases through inlet 106 with its outlet 105 directly, or via extension tubes, connected to a leaking ET tube. i.e. selected ET tube is smaller than what is ideal for that patient, like intubating a patient with a 4.0 mm tube where the ideal ET tube for that patient is a 6.0 or 8.0, for example. In this setting, acoustically energized and pressurized fresh gases enter patient's trachea and excess gas will scape airways through the leak. This form of acoustic ventilation, although very efficient and easy maintain and cheap to build, is imprecise as airway pressure monitoring is difficult.

In a similar embodiment of the invention and as seen in FIG. 5E which is the safest and most efficient form of acoustic ventilation, said acoustically energized flow of fresh gases exit sonic flue's outlet 105 and enter the inspiratory lumen of a double barrel ET tube DBETT. The expiratory gases exit through the expiratory lumen of DBETT and enter a modified air-hub 401-1, as seen in FIG. 5E, where a control valve (x) is maintaining a positive pressure. Dump (y) and limit (z) valves are also installed. All three valves are collectively marked as 408 in the figure. Sensors for pressure, temperature, and humidity are collectively marked as 404 in FIGS. 5D and 5E. The flue itself can have a limit valve (z) 411 for safety purposes. Pressure sensor of the air-hub has not been shown in FIG. 5C but measurement difference between this sensor and the pressure sensor on the flue can help in prompt recognition of obstructive issues inside the airways. The hub can also has a capnogram 409 and a sputum tray 407. Installation of ultrasonic transducer(s) 406 is optional as most of ultrasonic activation of airways come from ultrasonic flues as described earlier. In the setting of FIGS. 5D and 5E, pressure, temperature, humidity sensors 404 have to be installed on the walls of the hub. Same valves and sensors can be installed on the flue as well, in a fashion as to avoid acoustic interference as much as possible. Means for suctioning the expiratory lumen of the double barrel ET tube is also considered but not shown in the figure.

As seen in FIG. 6, square, sine, or triangle waves, pre-recorded sounds, especially designed harmonics, or computer generated tunes, etc. are created or played by a computer, wave generator circuitry 71, or similar devices. Said generated electronic signals are then amplified by an electronic amplifier 72 and then played by acoustic signal transducer(s) similar to a loudspeaker 73. Said transducer(s) can be made of coil on magnet, magnet on coil, coil on coil, magnetostrictive or piezoelectric technology. Generated acoustic vibrations with desired intensity and frequency or combination of frequencies are then introduced into patient's airways by means of a specially designed sonic waveguide/flue/tunnel/funnel/spiral/helix/cochlea/accelerator 80, directly or through the air hub 43.

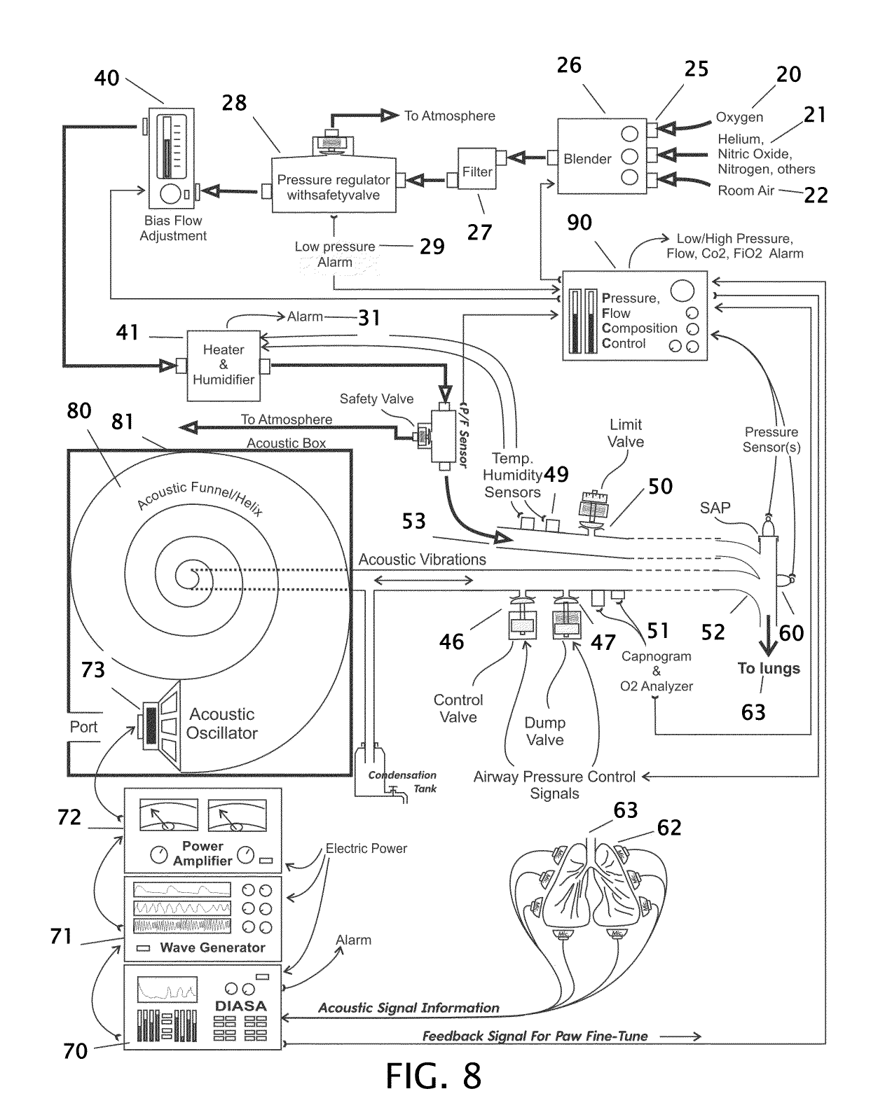

There are various combinations and multiple different ways of placing and setting the valves 46, 47, 50, sensors 48, 49, 51, patient air conduits 52, etc. on the air hub 43. In one embodiment, instead of air hub 43, inspiratory and expiratory limbs have been used, as shown in FIG. 8. Most of these sensors 48, 49, 51, and even valves 46, 47, 50 can be mounted onto the limbs or installed inside the main box of a ventilator machine and be connected to the air hub 43 or patient limbs of the machine by connecting tubes.

The acoustic vibrations entering air-hub/patient limb can be continuous, interrupted, fluctuating, musical and non-musical, synchronized in accordance with time in inspiration or expiration or non-synchronized. Selection of frequency range and intensity or loudness of the played vibrations in relation to time in inspiration/expiration will depend on patient's size and morphology, patient's medical condition, and wetness of airway/alveoli, thickness of secretions, pulmonary perfusion, etc.

Acoustic ventilation in its more complex form incorporates multiple automatic regulatory components at various stages and levels of the machine such as blending gases, flow adjustment, pressure control, heater/humidifier, etc. It also can incorporate a diagnostic and interventional apparatus called Diagnostic and Interventional Acoustic Signal Analyzer (DIASA) which will be discussed later.

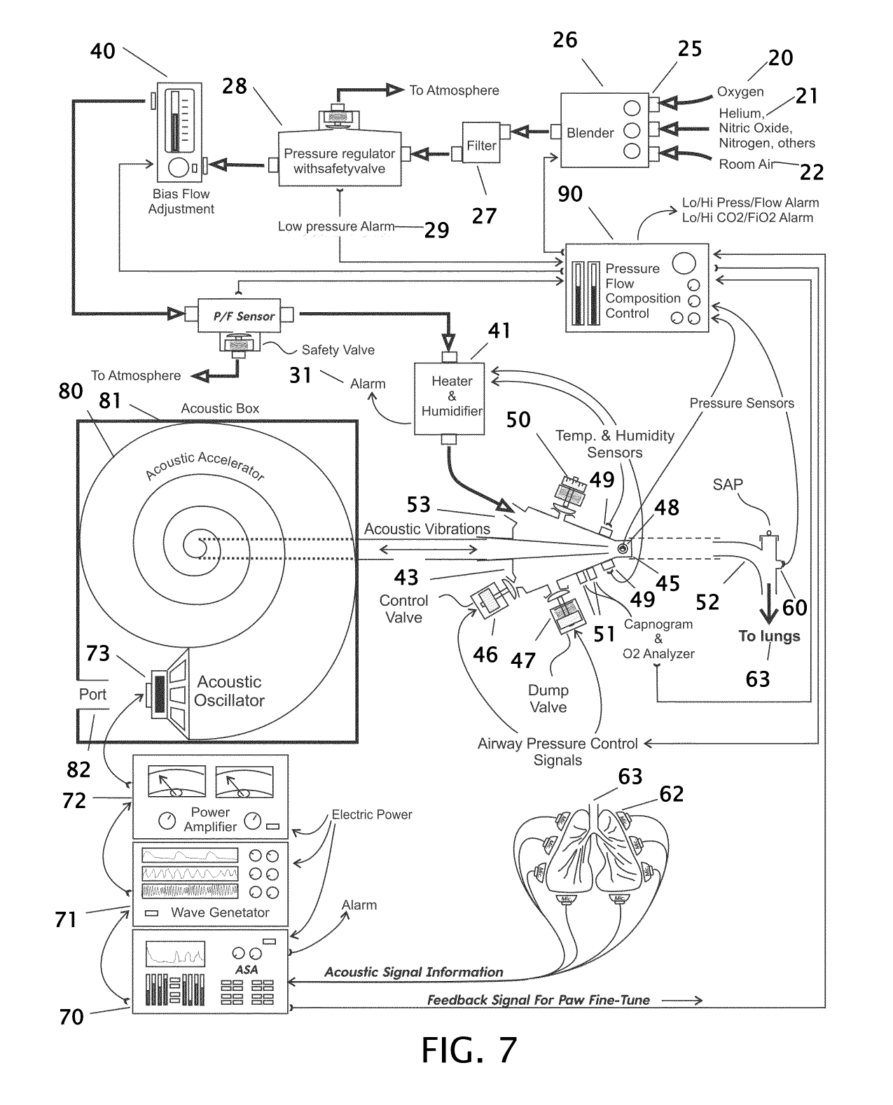

Various gas blender embodiments can be used with acoustic ventilation systems as described herein. Acoustic ventilation can work with simple pneumatic blenders such as the ones made by Bird® where there's no need for electricity, or it can work with electronic blenders some of them capable of doing all the mixing, flow adjusting, heating and humidifying of inhalational gases all in one unit such as Precision Flow® by Vapotherm. Acoustic ventilator can also work with an incorporated automatic electronic gas blending system, one embodiment of which is shown with an acoustic signal analyzer in FIG. 7. This embodiment shows a mixing of two or more gases in designated proportions in accordance with signals coming from gas pressure, flow, composition controller (PFCC) 90 which is an electronic module receiving signals from pressure sensor 48, O2 & CO2 sensors 51 and analyzes the data along with information coming from acoustic signal analyzer DIASA 100 and sends out signals to control valve 46 to set the pressure inside the hub and can also send signals to the automatic blender 26 and adjust mixture of gases and FiO². Solenoid controlled valves of such automated gas blenders and technologies to develop these machines already exist and are in use in the industry. In the setting of automated acoustic ventilation, PFCC is a computer software analyzing above mentioned data and making adjustments to said solenoid valves through an electronic interface. Although neither the software nor the interface exist today but its design involves simple engineering and available technologies. Acoustic ventilation is not dependent on this system and it works just fine through human interaction.

In another embodiment, shown in FIG. 8, the expiratory limb receives acoustic vibrations from the acoustic box 81 and delivers these vibrations into the patient's airways. Acoustic vibrations could also be introduced into the inspiratory limb, or into the hub 43 as depicted in FIG. 6-7. In FIGS. 6, 7, and 8, gas movements and directions are shown in bold lines and arrows in the schematic representations of acoustic ventilation.

Dump valve 47 can be opened when pressure exceeds a certain level. There is also a control valve 46 on the expiratory limb/hub 43 which is controlled by PFCC 90 and maintains positive airway pressure (PAP). The pressure is preferred to fluctuate in a manner similar to natural or exaggerated breathing called continuously variable positive airway pressure (CVPAP). As mentioned, dump valve 47 and control valve 46 are managed by Pressure-flow-composition controller PFCC 90 while safety valve 50 located on the hub or inspiratory or expiratory limb is merely mechanical and independent of the whole machine. It resembles safety valves used on ambu bags.

Pressure-flow-composition controller PFCC 90 is an electronic circuitry or a computer with software configured to receive information from pressure sensors 49, O2 analyzer 53, capnogram, and Acoustic Signal Analyzer (DIASA) 70, process the data, and subsequently adjust and maintain optimal pressure and composition of gases or FiO2 through controlling motorized bias flow meter 40 and automated blender 26. Said PFCC 90 can also adjust airway pressure to desired rhythm and variability in order to enhance and maximize alveolar ventilation with minimal risk of damaging the bronchial tree.

FIG. 7 shows an assembly of acoustic ventilation with DIASA which is composed of sonic receivers 62 resembling ordinary microphones with wider frequency range comparatively. They can be made of coil on magnet, coil on coil, magnet on coil, piezoelectric or similar technologies. The sonic receivers are placed on the chest of a patient in certain areas and constantly listen to the lungs just like a doctor examining a patient with a stethoscope. Sonic, infrasonic, and ultrasonic vibrations made by the acoustic ventilator's sonic flue enter patient airways and travel through lung parenchyma. These vibrations and the changes they go through during their travel thru lung tissue can be picked on a patient's chest wall skin by a stethoscope or by said sonic receivers. The sonic information picked will not only help in diagnosing certain pulmonary conditions but also can be used as a tool to monitor efficient ventilation. Data from sonic receivers 62 will be transmitted to DIASA 70 via wired or wireless technologies. Data containing information regarding changes in breath sounds and variations made to the oscillations during their travel thru the lungs and onto the chest wall will be analyzed in the central processing unit of DIASA which can simply be a computer with proper software. Accordingly, DIASA will detect areas of hyper-resonance, consolidation, or atelectasis and thereby adjust tones/tunes in frequencies and intensities. At the same time, DIASA will send feedback signals to PFCC 90 where an electronic or electromechanic interface can adjust pressure, flow, and FiO₂ accordingly. The simple or touch screen display of DIASA, like a computer's LCD, can show sonic return of each section of the lungs separately and help healthcare providers in detecting pathologies like pneumonias faster and easier. Since DIASA can also be used as a monitoring device, it is predicted that use of harmful x-rays as a tool to monitor any pulmonary pathology's progress or course will be reduced in future.

Major operations of PFCC 90, DIASA, and the wave generating circuitry can all be handled by a computer(s) loaded with proper software/applications. These interactive computer applications will control other automated areas of acoustic ventilator machine AVM, at times through mechanical and electromechanical interfaces. Human control will supersede and overwrite all operations, if needed.

In one particular form of DIASA, said electronic sonic receivers 62 also act as sonic and ultrasonic emitters to make the lungs transparent to eyes of a computer which is capable of ultrasonic imagery just like today's ordinary ultrasound machines. The information hence obtained is superior to simple x-ray in clarity/resolution and is easily comparable to a chest CT-scan in definition, with added advantage of continuous monitoring and without harmful radiation.

Telemetry can easily be applied to such setting for ICU, ER, OR, or even home use of AVM. Although the required software and computer applications, such as sound recognition, complex tone/tune generators, control panel for electromechanical interfaces, etc. do not exist today but one can understand that their development is easy and feasible nowadays. None of the technologies needed for a completely operational automated AVM is out of reach with today's technology.

Portable AVMs can work with desirable level of energy consumption, needed safety, and ease of use. Any medical grade compressor, air pump, blower, servo motor with proper turbine or fan can be applied to provide needed flow instead of the ordinary wall mounted gas supply. Roots-type blower might be a good choice as it can help in reducing both the size and power consumption of the ventilator although a bit noisy. Oxygen tanks can be used to increase FiO2 along with a simple light weight blender. These technologies already exist in the market. Pressure monitors, canpnograms, safety valves, etc. will remain the same as described in the document.

In a preferred embodiment of portable AVM, a blower sonic flue, called “Blower Bees” or “Beehive”, is used which can save in areas of power consumption, cost of manufacturing, and ease of use or handling as the device is relatively light, probably lighter than any portable ventilator machine currently available. At the time of design of this portable ventilator I was impressed by the way bees ventilate their hives and decided to call it as such. Blower Bees can be quite noisy and as explained earlier mufflers and filters can be used to reduce noise. In its simplest form, Beehives at time work without a blender and only filtered air is ventilated into the lungs which makes the machine very agile and perfect for simple transportation scenarios where supplemental oxygen is not needed such as a patient with headtrauma and intact cardiopulmonary integrity. In this simple form of portable AVM, there are means of providing added oxygen into the Beehive as discussed as shown in FIG. 9.

As seen in FIG. 6, simple tones, multiple harmonic or non-harmonic tunes and adjusts, or composed and recorded “pulmoryhthmic” or “pulmosonic” melodies are (1) played back or created and played by a computer or a tone generator 71, (2) amplified 72 and transduced to sound energy 73, and (3) delivered to our lungs 80.

An example of a pulmorhythmic music is an old electronic pop instrumental called “Popcorn” composed by Gershon Kingsley in 1969 which at least in parts is suitable for ventilating lungs. As odd as it may sound, these rhythms can truly boost our respiration and that is probably where our natural joy, passion, and desire for beats is coming from. Since the ancient warriors' drumming rituals till the modern upbeat pop or rock music, we just can't help but love it because it is intertwined with our livability. Today, there are thousands, if not millions, pieces of melodic beats and music with or without songs that you can play in one's lungs in the fashion molded by AVM, and sufficiently ventilate their lungs betterthan anycurrently available ventilator machine in the market or on R&D desk of any manufacturer anywhere in the world. Music to our lungs will be hailed soon.

Examples of “pulmosonic” tunes can be recorded sounds such as a baby crying or grunting, laughter, hiccups, sneezing, human voice singing or humming, bees' wings ventilating their hives, cats purring, crows' call, lullaby, rustling of leaves in the wind, waterfalls, etc. Combinations of different tones/tunes which are not necessarily rhythmic can also be used. These sounds should be able to create needed 3D cymatic effect on pulmonary non-Newtonian fluids such as sputum, pus, or blood and enhance their excretion out of lungs. Inspiratory/expiratory timing of these sounds and their intensity, duration, frequency range, etc. is also important. Number of pulmosonic tunes or sounds is practically limitless. Although these sounds miss the beat needed for “very high frequency ventilation” component of AVM but when combined with CMV style ventilation of AVM, they actually do a good job of opening alveoli, gently mobilizing secretions, and clearing airways. Combination of pulmosonics with pulmorhythmic will be the best setting for these machines.

Such abilities of AVM, elaborated in my previous patent “Respiratory Booster Machine, RBM” can be very helpful in disorders such as cystic fibrosis CF, ciliary dyskinesia PCD/Kartagener's syndrome, cerebral palsy CP, emphysema/COPD, and many other pulmonary or airway diseases. Especially designed tones/tunes with continuously variable frequency, usually moving from lower to higher pitch, can mobilize secretion very efficiently. Grunting is one such naturally occurring tune. Future work will pave the way for development and advancement of pulmosonics and pulmorhythmics.

The amplified tone or combination of tones/tunes are played by an electro-acoustic transducer 73 which delivers the pressure oscillations and sound wave energy onto the column of gas entering airways of a patient via a specially designed funnel 80 and tubing system. In some embodiments, the electro-acoustic transducer 73 can include a first solenoid attached to a cone and moving/vibrating back and forth in a magnetic field generated by a magnet or a second solenoid. Said electro-acoustic transducer 73 can also be made of piezoelectric crystal or magnetorestrictive technology, or a combination of all above mentioned techniques. The electro-acoustic transducer 73 blows pressure oscillations and sound vibrations into a funnel 80 which collects, preserves, and conducts the sound wave energy and pressure oscillations to the column of gas being delivered into the lungs of a patient via special tubes as discussed earlier.

The diameter of the acoustic tunnel/funnel 80 being gradually decreased in the direction of movement of sound waves in a fashion that sound wave energy reflections are preserved and effectively transferred to patient's airways. Cross section of said acoustic tunnel/funnel can be in any shape such as round, elliptical, square, rectangular, hexagonal, octagonal, etc. It can be bent, twisted, or folded onto itself in form of a progressively narrowing helix or maze so that it will take less space, be more compact, more efficient, easier to move, and contain inside a box that can also act as a sound barrier. In some embodiments, the acoustic flue is shaped like a spiral much like the shell of seashells or slugs or the cochlea of our inner ear, and therefore, it can be fitted inside a box which in turn can hamper and dampen the noise. This particular form of acoustic flue has amazingly about 20% more flow generated across the ET tube and works with less noise pollution if built properly. Although very light, but we should remember that breathable gases weigh something, albeit very little. Therefore, one can imagine that fast back and forth motion of gases inside the helix, almost at the speed of sound, pushes the air closer to the outer edge of the helix by centrifugal force and eases the way out the little hole at the end of the spiral, with least number of reflections and deflections. Porcelain or thick glass is a very desirable material in the context of performance but it has the disadvantage of heavy weight and being fragile. There are many substances that can be used in the make of acoustic or sonic flues as discussed earlier, hard plastics probably have many advantages as they can have smooth surfaces to reflect sound well, they are light, cheap, and easy to clean and manufacture. The color of the material probably does not matter at all but the inner surface of the flues better be so smooth that you can see reflection of your image in it, otherwise its output is less and its noise is more, albeit just a notch or more.

In one embodiment of acoustic ventilation the acoustic box can have an opening or port 82 in one side of the speaker box 81, preferably facing the rear of the electro acoustic transducer 73. The cross sectional shape, diameter, and length of the tunnel depends on the size of the acoustic transducer and the type of the acoustic ventilation needed, for example, ventilation of lungs in a larger subject needs larger oscillators and flues compared to smaller subjects who need smaller oscillators. In some embodiments, there is a second acoustic tunnel sealed and attached to the rear aspect of the oscillators in order to potentiate the sound waves in the first/front tunnel 80, not shown in drawings. The length of the first or second acoustic tunnel in some embodiments can be changed as the oscillator can move or slide back and forth inside the parallel part of acoustic tunnel, in a retractable or telescopic fashion or on railings designed for such purpose. In this way, the oscillator can be placed and adjusted and fixed at the sweet spot of the tunnel with the most power output. Variable length acoustic tunnel, through telescopic action, can be used for adjustments to achieve maximum acoustic power at the narrow end of the flue as well. In this setting, the walls of the acoustic tunnel, before they start to converge, can slide onto each other much like a telescope. These latter forms of acoustic flues are not shown in the drawings. The port 82 can be connected to a noise muffler 83 to reduce audible noise which is particularly loud in “Beehive” model of acoustic ventilators. The port 82 can be all closed to outer space of said acoustic box except in the “Beehive” model where the muffler also acts as a medical grade HEPA air-filter with connections for added oxygen as well, as shown in FIG. 9A. FIG. 9C shows a typical “Beewing” valve which can assume many shape or forms. These valves are very small, usually about 5-10 mm in diameter and many of them can be placed on the walls of the flue. They obviously attenuate the overall output of the flue but generate a flow toward the narrow or proximal end of the flue so the ventilator can operate without a source of compressed gas(s). The narrow lumen of these valves can be as long as 2-10 cm and they preferably should be installed on the inner curve of the acoustic accelerator/helix. Location of beewing valves on a linear or other types of sonic flues is not as important but generally it's preferred to install them closer to the oscillator. In one model, the beewing valves are set around the oscillator connecting the anterior to the posterior chamber, where posterior chamber can be connected to a filter/muffler and a source of oxygen. This version of sonic flue is mainly used in the portable models of AVM.

In some embodiments, electronic oscillations can be made by modulated wave-generating circuitry, computers, or simply any analog or digital device capable of playback such as an mp3/mp4 recorder/player, CD/DVD player, reel or cassette tape, records, or other similar devices. These electronic oscillations are then amplified and played by an electro-acoustic transducer 73. The played sonic/subsonic/ultrasonic vibrations, in the range of 3 Hz-50 KHz for example, then pass through said acoustic tunnel and the hub where positive pressure gases are introduced as discussed earlier and then via routes such as a regular or modified endotracheal tube, nasal prongs, facial mask, laryngeal mask, tracheostomy tube, or transcutaneously through the chest wall, or directly through the chest wall such as via a chest tube, etc. are delivered to the lungs 110 of a subject.

In yet another embodiment of the current invention, said transducers introduce the generated vibrations directly into a room such as emergency rooms' crash room, operating room, cabin of an ambulance or cabin of an aircraft where patients are kept/transferred, or into a chamber where a patient can be safely held, in order to help a patient, particularly an unconscious one, breath without a need for facial mask or endotracheal tube and alike. These especial types of oscillators employ very powerful and large acoustic transducers, as loud as 120-155 dB or more. These types of oscillators, not shown in drawings, can play vibrations that are not audible to people or medical staff as they can employ subsonic vibrations, in the range of 3-15 Hz for example, or a combination of audible and non-audible frequencies.

This type of “whole room acoustic ventilation” called “High Intensity Enclosure Ripples HIER” will not harm medical staff present or working in the room where the patient is located. These intense pressure fluctuations are well tolerated by humans although at times they give people an uneasy feeling in the ears or throat which most people will simply get used to it after few minutes. On any account, use of protective ear plugs is highly recommended as tympanic membranes are vulnerable to such direct impacts. Unfortunately, use of noise cancellation technologies of any kind would be counterproductive in this case.

Use of such “High Intensity Enclosure Ripples HIER” by a special type of AVM during transportation or in a crash room of an emergency department will give extra time to initiate more specific forms of PPV such as to intubate the patient, or it might obviate use of aggressive PPV. As mentioned earlier, the surface area of the diaphragm of HIER oscillators are significantly larger at around 1-2 feet in diameter depending on the size of the room. Similar oscillator technologies as discussed with other types of acoustic ventilation can be employed here. At the same time, use of piston and a direct servo motor to make the gigantic ripples might be a good alternative in the case of HIER AMV (not shown in the drawings).

The oscillator can be concealed inside the walls or above the ceiling or in an adjacent room or closet. This can be predicted in the structure of a building for a hospital for example, at the time of construction. It also can be accommodated at later time since some of these oscillators with their sonic flue can fit inside a space as small as a closet or few cabinets. The difference here would be the sonic flue's walls are not converging and instead they can stay parallel for the whole length of the waveguide. The width of such waveguide duct is same as the diameter of the oscillator at about 1-2 feet or 30-60 cm, and the length of the flue is about 18-24 feet. The room preferably should be relatively airtight and windows or instruments in the room should be secured as such strong vibrations can easily shatter a window or displace furniture or instruments that might resonate with the frequency. Healthcare personnel who will be present in the room

Said HIER acoustic ventilator not only helps an unconscious patient breath without any mask or ET tubes inserted in the airways but also can improve systemic and pulmonary blood circulation given our legs have valves in their veins and our hearts have 2 valves on either side which direct whole body pressure oscillation in one direction of liquid/blood flow. It may even be sufficient to keep vital organs irrigated with blood during a cardiopulmonary collapse from any reason except extreme hypovolemia or severe exsanguination. Therefore, HIER ventilation will play a major role in resuscitation of patients who might have had a cardiopulmonary arrest secondary to conduction disorders or arrhythmias for example. Obviously, since acoustic ventilation or in this context “acoustic resuscitation” with HIER has never been tested in humans or even animals as a resuscitative tool, studies to ascertain safety and efficacy have to be undertaken before such measures become standard of care.

Frequency of oscillations can vary from 3 Hz up to 25, although combination of added higher frequencies up to 250 might be beneficial. Higher frequencies generally are not effective or worth the noise pollution and service disruption. The intensities may vary from 100-150 dB depending on the enclosure size and shape and other elements. In one study, lungs of anesthetized dogs were sufficiently ventilated so they did not have to breathe when exposed to acoustic vibrations of 7 Hz at about 135 dB.

Another method of acoustic ventilation with HIER is use of revolving leaves or blades. Usually four or more large rigid leaves rotating about a central vertical pivot, installed inside a large cylinder with openings on either sides, much similar to revolving doors of an office building. The whole assembly can be laid horizontally as well, depending on other elements. Each leaf or blade can be as big as few feet on length and width. Rapid revolution of a blades can generate acoustic vibration with intensities as high as 110-130 at around 3-15 Hz. An electric motor will rotate the blades at a pace or frequency controlled by a control panel. Again, the larger the subject to be ventilated or resuscitated, the lower the frequency needed to be generated, and vice versa. The intensity of the ripples can be adjusted by the size of the blades or openings on either side of the encasing cylinder. First opening of said cylinder will be inside the enclosure where the patient is located and second opening is in a an enclosure similar to a closet of a box containing the contraption or an adjacent room with desired temperature and humidity as the air in the two rooms will constantly exchange. The second room does not have to be as big as the first room.

In one special type of HIER, a larger size modified Roots-type compressor will generate the tides. In this particular type a duct has to feed the rear port of the compressor with same room air otherwise, air from other rooms or outside the building has to be constantly pumped into the room. Number of blades differs in various models of the HIER cardiopulmonary resuscitation system. In the simplest model there is only one blade swinging back and forth against a fixed wall or another immobile blade and generates a ripple and sending it directly inside the room or enclosure where the patient is located, or sending the ripple into a resonating waveguide with a length suitable to desired frequencies usually from few feet to few yards. Said clapping blades are installed on a stable platform that can move on a guiderail back and forth inside the waveguide. Multiple blade revolving door style of the subsonic wave generator can also be made where the blades almost hit the immediately preceding one as they approach the outlet to help compress the air between the two blades and send a strong ripple into the patient enclosure. In this case, the blades are not fixed to the pivot at all times and while the pivot is constantly rotating the blades in proximity of patient enclosure will have a pause awaiting for the following blade to slap it with compressed air and push it back on its allocated fitting spot on the pivot where the constantly rotating pivot will pull it back into the order the blades are distanced from each other on the distal part of the assembly till next clapping incident when the blade will be temporarily unhooked from the pivot. A simple form of such assembly will look almost like the old desktop revolving Kardex carousels when cards fall on top of each other when a horizontal pivot is rotated around. It is not hard to imagine with fall of a card on top of the previous one a tiny tiny ripple is made. Another example of a sonic vibration maker suitable for non-invasive resuscitation in the manner discussed is a large scale rotary siren or air cutter. The air cutter has to stay below 20 Hz with large enough blades to generate ripples as strong as about 90-140 dB. These ripples can be amplified by a resonating waveguide as mentioned. The waveguide can be winding in order to save space.

In one embodiment of HIER assembly, there are two opposing blades swinging about an axis and driven toward and away from each other few times per second, just like a bird flying, more like a hummingbird, or someone clapping really hard and really fast. In one model, one of the blades/leaves is stationary while the second one is swinging few times per second. Again, these blades are quite large and bulky, at times 2×4 feet or even larger. In one assembly of current invention, said leaves are installed inside a duct big enough to contain the leaves. The duct which can be as long as 40 feet or even longer will act as a waveguide and potentiate the ripples before they enter the enclosure where the patient is being treated. In another embodiment, a larger modified Francis turbine generates the powerful ripples and a duct will guide the waves into the enclosure. This will almost resemble the rotary air cutter siren style discussed. A modified Pelton turbine can also do the job. It is not out of reach to vibrate a whole modified wall on one side of the enclosure to generate the much needed vibration to ventilate the lungs or resuscitate a person. Such modified walls will act like a giant oscillator where a servo motor or a larger coil on magnet can vibrate them enough. Magnetorestriction without a lever does not seem to bring powerful enough low frequency ripples. Piezoelectric is generally unsuitable unless a battery of the crystals are used. Another name for HIER AVM can aptly be extracorporeal blood circulator which at times 2 or more of these machines can send multidirectional ripples into a patient's body and sustain blood circulation along with respirations.

Acoustic ventilator can be configured to generate and transmit particularly designed and timely delivered vibrations and pressure oscillations to the lungs 110 of a subject to ventilate, improve ventilation, augment diffusion of gases across alveolar membrane, prevent focal alveolar collapse and/or atelectasis, enhance effective expectoration, advance even distribution of medications administered via trachea, and other yet to be measured effects such as assistance with pulmonary perfusion.

As mentioned earlier, regular AVM has an acoustic signal generator 71 having modulated wave-generating circuitry and an acoustic signal transmitter 72 having an electro-acoustic transducer 73, and as explained above, waves generated by a computer or a wave generator circuitry 71 are amplified by a power amplifier 72 or the like and sent to an electro-acoustic transducer 73 or sound wave player sealed onto the inlet of a similar size funnel 80 made of metal/plastic/glass or similar material. The narrow end or the outlet of said funnel 80 is connected to a tube 70 which guides the generated and amplified sonic, subsonic, ultrasonic waves and pressure oscillations into patients' airways 60 directly or through a specially designed Y-connector or a hub. The hub 52 has another inlet 54 for a precisely measured bias flow of desired mixture of heated and humidified gases with precisely measured airway pressure as shown in FIG. 1. The hub 52 also has an outlet 55 for escape of the gas with a valve precisely controlling the airway pressure, and also few attachment locations for pressure sensors, alarm sensors, and a mechanical pressure safety valve 50.

The sine/square/triangle waves in the range of 3 Hz-50 KHz are created by a computer or an electronic pulse generator circuitry 71 which is connected to a power amplifier 72. The amplified tone or combination of tones then are played by an electro-acoustic transducer 73 similar to a loudspeaker system which delivers the pressure oscillations and sound wave energy onto the column of gas entering airways of a patient via a specially designed funnel 80 and tubing system.

In some embodiments, the electro-acoustic transducer 73 can include a solenoid attached to a cone, just like a weather proof loudspeaker, woofer, or subwoofer, and moving/vibrating back and forth in a magnetic field generated by a magnet or another solenoid. The electro-acoustic transducer 73 can also be made of piezoelectric crystal technology which is quite efficient particularly in higher frequencies and have the advantage of smaller size, lighter weight, and not necessarily require a magnet/solenoid and therefore, is safe to use even adjacent to an MRI machine.

The electro-acoustic transducer 73 as shown, for example in FIG. 6, blows the pressure oscillations and sound vibrations into a funnel 80 which collects, preserves, and conducts the sound wave energy and pressure oscillations to the column of gas being delivered into the lungs 63 of a patient via flexible tubes 60. Because majority of AV systems are dependent on electric power, a set of rechargeable batteries can seamlessly continue to power the system for hours in case of a power outage, not shown in the drawings. There are alarm systems incorporated, as to notify personnel of the power outage and/or amount of available battery power or remaining time before system shut down. Similar battery pack can be utilized in portable models of acoustic ventilation.

As previously described, the diameter of said funnel/tunnel 80 can be gradually decreased in the direction of movement of sound waves in a fashion that sound wave energy reflections are preserved and effectively transferred to patient's airways. This particular type of funnel 80 can take a lot of space as it is very long but in one of its forms it is designed and shaped like a spiral much like the shell of seashell or slugs or the cochlea of our inner ear, and therefore, it can be fitted inside a box 81 which in turn can hamper and dampen the noise. In one embodiment the box 81 is designed similar to a loudspeaker or subwoofer enclosure. The box 81 has an opening or port 82 in one side of the speaker box 81, preferably facing the rear of the electro acoustic transducer 73 the port 82 is connected to a noise muffler 83 much like a car muffler 83. The port 82 can also be used to be connected to the funnel 80 and tubing assembly through which vibrations will be delivered to patient airways.

In another embodiment there is a set of noise cancellation system composed of one or more sensor(s) or microphone(s) to pick up environmental noises, process them by a noise cancellation circuitry (already available technology), amplify created responsive noise cancellation electronic signals, and then connect the amplified signals at desired power level to a set of loudspeaker(s) capable to playing needed range of frequencies and amplitudes sufficient to help reduce unwanted noise in areas where the machine is used. Noise cancellation technology is affordable, accessible, and has been used for many years, although to the Applicant's knowledge not in conjunction with mechanical ventilation technology. These embodiments are shown in FIG. 9, showing two methods of noise reduction with acoustic ventilation and or a respiratory booster machine.

In yet another embodiment the sonic flue or funnel 80 is not as long as jam's equation estimation, or there is no helix/spiral/vortex made out of the funnel 80 although sound energy deliverance might be less than the longer spiral cochlear forms and operational noise higher, but the machine may still work, albeit at a less than desired ability or expectation.

Electronic oscillations can be made by modulated wave-generating circuitry 71, computers, or simply any device capable of playback such as an mp3/mp4 recorder/player, CD/DVD player, reel or cassette tape, records, or other similar devices. These electronic oscillations are played by an electro-acoustic transducer 73 or acoustic signal generator which is comparable to a regular or modified weather-proof loudspeaker 73, the played acoustic vibrations then are delivered to the lungs of a subject via routes such as a regular or modified endotracheal tube, nasal prongs, facial mask, laryngeal mask, tracheostomy tube, or directly through the chest wall, and so on. Supplemental transducers can be used to increase the pressure or frequency range within the funnel 80, and/or to provide sound cancelling as to reduce external audible noise. In some embodiments, said acoustic tunnel is built in easily separable parts, such as two opposing polarities, in order to make cleaning and disinfection easier.

Acoustic ventilator can be configured to generate and transmit particularly designed and timely delivered vibrations and pressure oscillations to the lungs 63 of a subject to ventilate, improve ventilation, augment diffusion of gases across alveolar membrane, prevent focal alveolar collapse and/or atelectasis, enhance effective expectoration, advance even distribution of medications administered via trachea, and other yet to be measured effects such as assistance with pulmonary perfusion.

The machine has an acoustic signal generator 71 having modulated wave-generating circuitry and an acoustic signal transmitter having an electro-acoustic transducer as explained above. Vibrations from the acoustic signal generator are delivered into the subject's trachea and bronchial tree via a specially designed hub and tube system where a precisely measured flow of mixed desirable gases are introduced and then delivered into the endotracheal tube, respiratory mask, nasal prong, or similar devices into patient's lungs. The tubing system has an air hub 43 with sites for tubing connections and areas for installation of safety valves, pressure alarms, gas analysis, and other necessary sensors. There is also an outlet 45 which allows the mixture of gases enter patient circuits. In some embodiments, there is a diaphragm made of silicon, rubber, plastic, or the like which acts as a barrier between the oscillator or acoustic tunnel/funnel and the rest of the tubing system in order to avoid moisture condensation inside the funnel or in vicinity of the oscillator.

Referring to FIG. 6, the first of the major air hub connections 42 is an inlet which allows a precisely measured pressure and flow of a mixture of desired gas(es) to enter the hub. Said hub having another inlet to allow the acoustic vibrations to enter the hub 43, and another outlet 45 which is to be connected to patient's endotracheal tube or similar devices to deliver the desired gas mixture with adjusted pressures and pressure oscillations, energized with sound waves, into patient's airways and down the bronchial tree. The hub 43 is equipped with sensors 48, 49 measuring pressure/temp./humidity and even in some models indicators for composition of gas mixture in the hub such as a capnogram and O2 analyzer. Extra openings/ports for suctioning or attachment of other sensors/devices can also be accommodated. The excretions tray is not shown in this figure. Please note that the hub components are numbered differently in FIG. 5.

There are safety valves and pressure/flow alarms, similar to HFOVs, connected to the hub or nearby tubing system which constantly monitor optimal gas pressure fluctuations and prevents undesirable pressures/oscillations in case of a machine malfunction or added pressure or resistance by patient's spontaneous breathing efforts. These sensors are also capable of reporting gas leakage in/around ET tube and so on, just like some CMVs and HFVs. In some models of AVM, gas leakage around ET tube is a desired feature.

In one embodiment the acoustic signal receivers 62, similar to microphones, are placed on certain locations on the chest wall of the patient. These acoustic sensors 63 which can all be installed on certain spots on a patient's chest similar of EKG electrodes or similar fashion can detect frequencies of various ranges, in some models even detecting higher range of ultrasound even as high as 2-10 MHz. Some models can have acoustic signal sensors 62 capable of emitting ultrasound and reading reflected sonic waves or rays similar to an imaging ultrasound machine. Sensors 62 are connected to a computer and specially designed software constantly listening to sounds emitted by the patient's lungs, just like a physician listening to a patient's lungs by a stethoscope, and analyze sounds/noises emanated from the lungs. Auditory information is constantly analyzed and processed by the machine's computer, depicted as “Acoustic Signal Analyzer, ASA” in FIG. 7, also called “diagnostic and interventional acoustic signal analyzer, DIASA” which is configured to detect pulmonary pathology such as hyperinflation or consolidation areas and accordingly adjust AVMs frequencies, waveforms, wave power or intensities, pressures, bias flow, gas mixture, PAP or CVPAP, etc. which will enable AV to homogenously ventilate lungs and optimally reduce atelectasis, with the least amount of pressure possible, mostly without need for human interaction. Apparently, acoustic vibrations made by AVM and introduced to main bronchus of each lung travels through lung tissue and reaches the chest wall. These frequencies go through different destinies and change character in intensity of their various frequencies when there is blockages of airway by mucus plugging for example or if there is an abscess or areas of consolidation or hyperaeration and so on. The character changes these sonic vibrations go through can serve as a tool to monitor machine's performance and make needed adjustment, and at the same time, diagnose pathology. Voice/sound recognition technologies have advanced significantly in recent year and it is tangible to write software capable of analyzing pulmonary sounds to enable the machine to automatically adjust itself and alert personnel of need for human interaction. Currently, various sonic receivers and ways to develop such software with required electromechanical interfaces, etc. are being investigated. Teams of sound engineers, electronic engineers, computer engineers (software only), are to get formed to handle the details of practical aspects of the design. It is all about implementing needed technologies, which are all currently available, and polishing a new technique of ventilating lungs.

In one embodiment of the acoustic ventilator, such lobar ventilation monitoring can be achieved by means of ultrasound which is emitted by the acoustic flue or peripheral acoustic sensors 62, This will enable us to estimate overall aeration of various parts of lungs or lobes. The acoustic signal receivers or microphones 62 can be attached to the patient's chest wall by, e.g. adhesives, or mounted inside a modified jacket which can be put on the patient's chest. The acoustic signal analyzer will process the received acoustic signals from the patient's thorax and is configured to make adjustments in machine's setting and can also detect detachment of sensors 62 from the chest wall and warn care givers.

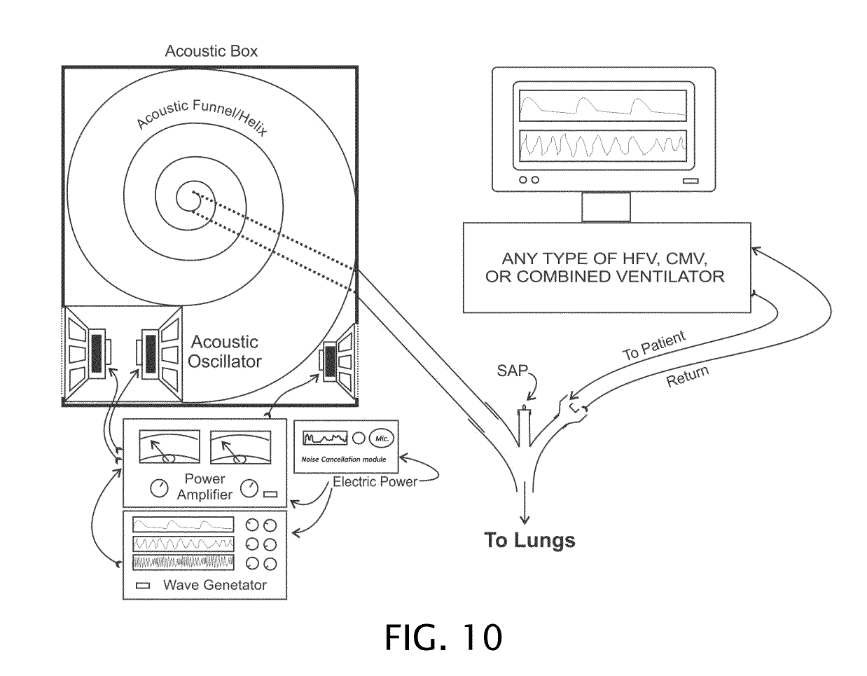

Acoustic ventilation can also be coupled with and work in unison with various CMVs, HFOV, HFJV, Bubble CPAP, HFPV, and other types of ventilation. It's best companion though would probably be a BCV, together they will have least VILI possible. Cooperative function of AV with another types of ventilator machines is shown and described in FIG. 10.

It is an object of the acoustic ventilation and respiratory booster machine to show desirable effects of sound energy on any column of gas in tubular and non-tubular structures, enabling the column of gas to resonate any tissue or material down the path including airway secretions and eventually alveolar membranes and therefore, facilitating pulmonary toilet and increasing active gas diffusion surface area.

It is an object of the acoustic ventilation and respiratory booster machine to use Invasive and non-invasive artificial ventilation with combinations of higher than high frequency or very high frequency ventilation (VHFV) in the range of 20 Hz-20 KHz, and ultrahigh frequency ventilation (UHFV) in the range of 20 KHz-10 MHz plus CMV mode and HFOV mode. Combination of said modes of ventilation with higher frequency vibrations can be very helpful in certain conditions particularly premature babies with super delicate airways. Administration of VHF and UHF vibrations on top of CMV+HFOV can boost respiration and lower the need for required FIO² and Paw, which is very desirable in reducing ventilation associated lung injury VILI.

In some embodiments, it is an object of the acoustic ventilation and respiratory booster machine to be available with sonic oscillators in the ranges of, for example, 3-250 Hz, 250 Hz-1.5 KHz, 1.5-8 KHz, 8-50 KHz and a powerful ventilatory command, particularly in the range of 15 Hz-75 Hz.

In some embodiments, it is an object of the acoustic ventilation and respiratory booster machine to be used with a 10 Hz-10 KHz sine/square/triangle tone generator connected to an electronic power amplifier driving an electro-acoustic transducer capable of carrying out varied intensities of the amplified tones. The oscillating transducer is connected to patient lungs through a specially designed funnel and non-compliant but flexible plastic tubing. Oscillations are mainly in the range of 10 Hz-500 Hz (600-30,000/min) in some of the preliminary models of the oscillating assembly.

In some embodiments, a catheter with an electrode at the tip is placed in the patient's esophagus which collectively with its circuitry detects phrenic nerve activity and will be able to temporarily mute/pause the ventilator and allow patient to breathe on their own drive. Other feedback mechanisms to temporarily mute/pause or otherwise adjust the ventilator upon the sensing of one or more parameters are also contemplated.

It is an object of the acoustic ventilation and respiratory booster machine to operate with minute volumes as high as 15 L/min, 20 L/min, or more, generated with 15-75 Hz with sine/square/triangle/saw tooth waves.

In some embodiments, nebulized steroids, bronchodilators, antibiotics, insulin, or other therapeutic or experimental agents, instilled, sprayed, or nebulized into a sonically charged column of air can produce a much more evenly distributed administration compared to current technologies. Pressured gas or ultrasonic nebulizers can easily be incorporated into design of AVMs air hub or patient circuits, not shown in the figures. In one embodiment of the invention, slow drip of surfactant or any other liquid or dry powder medicine (such as Budesonide, for example) in proximity of tip of sonic flue 105, or air hub flue 403 can disperse and nebulize the medicines very quickly. This was shown during in-vitro tests of the machine. Dispersion of surfactant into various parts of lungs will be done evenly, and much faster.

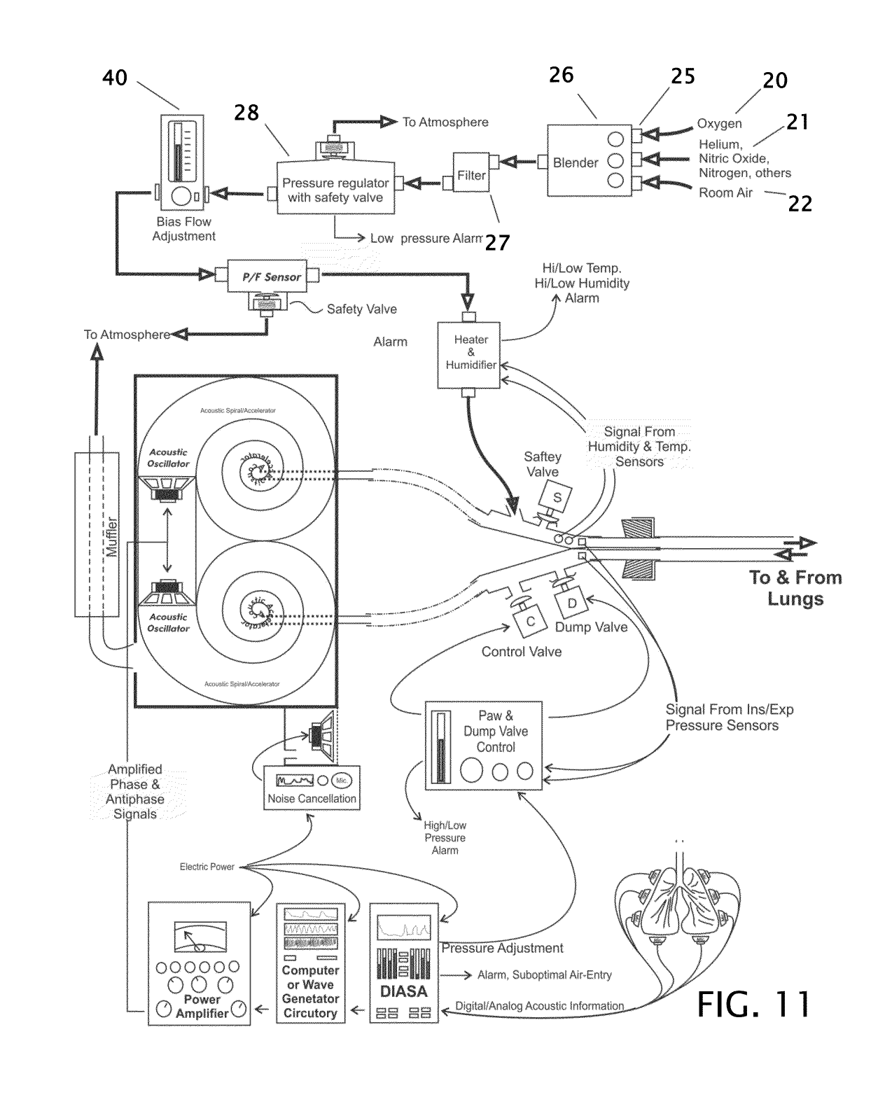

In another embodiment an acoustic wave player not only helps the flow of gas into the airways but also works as a noise cancellation device. This particular type of dual acoustic wave player will facilitate active expirations and can be used with a dual lumen ET tube. FIG. 11 shows a schematic presentation of a twin flue acoustic ventilation TFAV system, also called double barrel DBAV, having a first and a second set of acoustic generator and transducer with corresponding first and second spiral or helical sonic flues, or any other types of acoustic flues mentioned earlier, and a double barrel endotracheal tube. The dual barrel acoustic ventilation device would operate with a double barrel endotracheal tube. FIGS. 11 and 12 show some embodiments of double barrel endotracheal tube variations having a plurality of lumens. One lumen is for inspiratory gases and one for expiratory gases, passive expectoration, and constant pulmonary toilet without the need to suction patient's airways. As illustrated in FIG. 12, the first inspiratory lumen and the second expiratory lumen could be side-by-side lumens, concentric lumens with the inspiratory lumen being an inner or an outer lumen with respect to the expiratory lumen, a lumen having one, two, or more linear and/or curved inner walls traversing the inner diameter of the lumen and configured to divide the lumen into two, three, or more zones. In some embodiments, there could be one inspiratory zone and two expiratory zones, or vice versa. In some embodiments, a cross-sectional area of the inspiratory zone could be greater than or less than that of a cross-sectional area of the expiratory zone at the same cross-sectional level.

Tip of ET tube and its rim has a particular importance in generating effective turbulence and depth of penetration of fresh gases into smaller airways. The tip in some ET tube models can have a slightly thicker rim or a tiny ridge at the end, albeit seemingly counterproductive, as it narrows the lumen a notch but it might help in another way and even overcompensate for such disadvantage. As seen in FIG. 12 A₁, when the air is being pushed down the tube a tiny negative pressure area will be created right behind the ridge and therefore, make the passing column of gas to roll onto itself and make a doughnut shaped pack of gas moving down the airway. There are many toys (such as Airzooka®) that act in similar way. Doughnut packing and shipment of air can send the fresh gases deeper down the airways and defy dead-space volume which can be as high as 150 mL in adult humans. Said ET tube outlet ridge, also called “spout ridge” has never been employed in any type of ventilation systems. The speed, pressure, and duration of passage of gases/air over the ridge is important as to create needed level of negative pressure and form the gas doughnuts. As a general rule, the more explosive the flow of inspiratory gases the smaller the size of needed spout ridge, and vice versa. Therefore, HFJV probably needs a smaller spout ridge compared to HFOV, because inspiratory gases move faster and in a shorter period of time. It is postulated that CMVs are not suitable to work with spout ridged ET tubes unless the inspiratory gases are introduced in a pack of smaller gas infusions. That means, we should design a new type of CMV that instead of giving a 500 mL breath over 2 seconds, can give 10 inspiratory blows each of them 50 mL, each blown into the trachea over 0.1 second, with 0.1 second pause between each sub-breath. For many years I have been thinking we should modify HFJVs to work in such manner. Obviously, expiration time in this case will be about 3 seconds, given if we are going to give 12 breaths per minute. Such tiny explosive sub-breaths will generate a strong far reaching doughnut pack of fresh gases that can travel deep into smaller bronchioles with the help of a tiny spout ridge. This design will make a hybrid CMV/HFJV which might work better than either CMV or HFJV. In acoustic ventilation, since inspiratory flow of gases are way faster than any other ventilation methods, the rim of any ET tube is sufficient to generate enough turbulence to generate micro doughnuts in the stream of fresh gases. Although penetration of gases into even the smallest bronchioles is unsurpassed with ordinary ET tubes, or DBETT, but still a very small spout ridge, or slight modification to the rim of the proximal end of any type of ET tube used, will make a notable difference. Although shape and form of spouts have been studies and used extensively in other industries but unfortunately, this important factor has been left in limbo in art of ventilation. With advent of AV and inadvertent future improvements in total parenteral nutrition (TPN, PPN, etc.) it is quite possible that premature babies born as young as 20 weeks gestational age might survive. Clinical outcome of premature babies older than 23-24 weeks gestational age will drastically improve with emergence of acoustic ventilation in practice of Medicine.

The concept behind AVM is very simple and it has been a matter of unyielding astonishment to the inventor how effects of sound on respiration was left unnoticed in Medicine and how sonic flues were never properly explained in Physics.

Simply explained, acoustic ventilator's service is composed of 7 stages. As seen in FIG. 6, Tones, tunes, or composed melodies (pulmoryhthmics and pulmosonic) are played back or created and played by a computer or a tone generator 71 (stage1), amplified 72 and transduced to sound energy 73 (stage2), packed and delivered to our lungs by sonic flue 80 (stage3) or the most important stage of the invention. Stage (4) will employ a flow of heated humidified with desired blend of gases 26-41, brought to stage (5) which is pressure controlled mixing of inspiratory and expiratory gases in the hub 43. Stage (6), is stimulating airway gases with sound energy 408 & 409 of FIG. 5 for example. Final stage of AVM's gift, stage (7), is continuous monitoring and automated respiratory management by DIASA 70 & PFCC 90.

Acoustic ventilators AV, also called sonic ventilators SV or sound block ventilators SBV, utilize less energy to produce required waves and can operate for long hours on batteries. They are quite portable and user friendly. Acoustic ventilation technology is affordable (US manufacturing cost range from about $5000 for simple models up to more than $100,000 for more sophisticated fully automated models with ultrasonic capabilities). Although operation of these machines is based on sound energy but they happen to be working quieter than most HFVs, albeit they are a bit noisier than CMVs.

Based on its unique sonic waveguide design, current invention generates significant oscillatory gas diffusion at the tip of even smallest ET-Tubes, without dependence on pressure. Acoustic ventilators can run on batteries for many hours, making them ideal for transportation. Generated flow can amazingly be transferred for long distances inside a suitable type of tube as this ventilation technique is based on sonic vibrations and sound can travel easily for distances inside tubes.

Mechanically focused sonic energy has not received justified attention. Advent of infrasonic, sonic, and ultrasonic flues will open a Pandora box of possibilities in medical and other industries. Sound may have been the scene setter for development of life on this planet. Acoustic flues may pave the way to a justified appreciation of effects of sound in nature, still and alive.

In this document multiple embodiments of an acoustic ventilation system and respiratory booster machine have been disclosed. It should be apparent, however, to those skilled in the art that many more modifications besides those described are possible without departing from the inventive concepts herein. The inventive subject matter, therefore, is not to be restricted.

The terms “approximately”, “about”, and “substantially” as used herein represent an amount close to the stated amount that still performs a desired function or achieves a desired result. For example, the terms “approximately”, “about”, and “substantially” may refer to an amount that is within less than 10% of, within less than 5% of, within less than 1% of, within less than 0.1% of, and within less than 0.01% of the stated amount. Although certain embodiments of the disclosure have been described in detail, certain variations and modifications will be apparent to those skilled in the art, including embodiments that do not provide all the features and benefits described herein. It will be understood by those skilled in the art that the present disclosure extends beyond the specifically disclosed embodiments to other alternative or additional embodiments and/or uses and obvious modifications and equivalents thereof. In addition, while a number of variations have been shown and described in varying detail, other modifications, which are within the scope of the present disclosure, will be readily apparent to those of skill in the art based upon this disclosure. It is also contemplated that various combinations or subcombinations of the specific features and aspects of the embodiments may be made and still fall within the scope of the present disclosure. Accordingly, it should be understood that various features and aspects of the disclosed embodiments can be combined with or substituted for one another in order to form varying modes of the present disclosure. Thus, it is intended that the scope of the present disclosure herein disclosed should not be limited by the particular disclosed embodiments described above.

As mentioned earlier, AV/SV/SBV can be very helpful in patients with cystic fibrosis, asthma, chronic obstructive pulmonary disease COPD, respiratory distress syndrome RDS/ARDS/IRDS, bronchiectasis, cyanotic and non-cyanotic cardiac disorders, and some other cardiac and pulmonary conditions. AV can bring the extra edge in the science of ventilation of lungs. Sonic ventilation with acoustic signal analyzer (ASA) will not only make pulmonary care much more accurate but also free up personnel time. Outcome of very low birth weight babies will definitely improve with the new method of ventilation.

Advantages of Acoustic Ventilation:

Acoustic ventilation utilizes lower pressures, up to 50% less compared to other techniques. It also can lower FiO₂ requirements, and as such lower caustic effects of oxygen in our lungs. On the other hand, it clears lungs off unwanted excretions much faster, and therefore can reduce time on artificial ventilation. These three elements, pressure, FiO₂, and duration of ventilation are the major predictors of VILI, all three improved by AVM. The machine can be used independently or in combination and cooperatively with any type of ventilator machine where acoustic ventilator becomes respiratory booster machine (RBM).

There is less concern with patients' spontaneous breathing or “patient fighting machine” as pressure fluctuations do not interfere with the machine's performance or hurt the patient. Weaning patients off the ventilator is also easier with AV as continuation of vibrations do not interfere with patient's spontaneous breathing. A totally healthy and breathing subject can utilize AV, for enhanced performance or mucus clearing for example, without any interference with respirations. Simple models of AVM, particularly infrasonic one, can be aptly used for sleep apnea.

A major drawback of AV is its loud operational noise in some models since a major part of its frequencies lay in lower audible tones but on any account this type of noise at times can be very soothing indeed, to the patient and/or staff. Ambience auditory suppression and/or noise cancellation technologies can be installed adjacent to the ventilator or as an integral part of it in order to dampen the noise production, including use of mechanical technologies such as mufflers 83 as shown in FIG. 9A, or electronic noise cancelling modules 75 such as illustrated in FIG. 9B. Newer prototype models built in 2013, in accordance with Jam's equation for sonic flues, were much quieter with operational noise levels of only 58-67 dB without any particular muffler or noise cancellation system. It is anticipated that sonic flues will be very quiet in deed when manufactured with quality material and proper acoustic insulations. They still can remain as loud as required, but only inside the airways.

Another exciting prospect of AV is its inherent ability to open collapsed alveoli with lower pressures. Sound energy in proper frequencies travels readily down the smallest airways and even end-bronchiols and can vibrate and open collapsed alveoli with less pressure compared to currently available technologies. A similar spectacle happens every day in nature when a newborn's cry with first few breaths, pushes positively pressured air charged with sonic vibrations further down smaller airways and recruits more alveoli which inherently resonate with vocal range vibrations. It is mind boggling how this very simple and primitive part of pulmonary physiology remained mainly unnoticed for such a long time, lungs do better with sound in them. Natural simple phenomenon of boosting breath with sound, can be artificially reproduced and used for the benefit of an improved respiration in health and sickness, through embodiments as described herein.

Thus, specific embodiments of an acoustic ventilation and respiratory booster machine have been disclosed. It should be apparent, however, to those skilled in the art that many more modifications besides those described are possible without departing from the inventive concepts herein. The inventive subject matter, therefore, is not to be restricted except in the spirit of the appended claims.

### REFERENCES

[1] Didier Dreyfuss and Georges Saumon “Ventilator-induced Lung Injury”, American journal of Respiratory and Critical Care Medicine, Vol. 157, No. 1 (1998), pp. 294-323. doi: 10.1164/ajrccm.157.1.9604014
[2] Broner, N, The Effects Of Low Frequency Noise On People—A Review, journal Of Sound And Vibration 58 (4), England, 1993.
[3] Gavreau, Vladimir, Infrasound, Science journal 4 (1), USA, 1968
[4] Ter Haar, Gail; Coussios, Constantin (2007). “High Intensity Focused Ultrasound: Past, present and future”. International journal of Hyperthermia 23 (2): 85-7. doi:10.1080/02656730601185924. PMID 17578334.
[5] A. Vladi§auskas, L. jakevidus. “Absorption of ultrasonic waves in air” ISSN 1392-2114 ULTRAGARSAS, Nr.1(50). 2004.
[6] Norris Elwood, “Parametric ring emitter” U.S. Pat. No. 7,088,830.
[7] Pompei, F Joseph. “Directed acoustic sound system”, U.S. Pat. No. 8,538,036.
[8] Eva B. Holmberg, Robert E. Hillman, and Joseph S. Perkell.“Glottal airflow and transglottal air pressure measurements for male and female speakers in soft, normal, and loud voice”. J. Acoust. Soc. Am. 84, 511 (1988); http://dx.doi.org/10.1121/1.396829
[9] J A Fiz, A Carreres, A Rosell , J M Montserrat, J Ruiz, J M Morera. “Measurement of maximal expiratory pressure”. Thorax 1992;47:961-963 doi:10.1136/thx.47.11.961
[10] T Yazakil, Y Tashiro, and T Biwa. “Measurements of sound propagation in narrow tubes”. Proc. R. Soc. A 8 Nov. 2007 vol. 463 no. 2087 2855-2862
[11] Bo Wen, “Modeling the Nonlinear Active Cochlea, Mathematics and Analog VLSI”, http://www.stanford.edu/group/brainsinsilicon/documents/06_Thesis_BoWen.pdf
[12] D. Manoussaki, E. Dimitriadis and R. S. Chadwick. The cochlea's graded curvature effect on low frequency waves, Physical Review Letters 96, 088701 (issue of Mar. 3, 2006).

1. A sonic flue and an acoustic ventilation system comprising:
an acoustic signal generator;
an acoustic signal transmitter operably connected to the acoustic signal generator;
the acoustic signal transmitter having a first acoustic transducer;
the first acoustic transducer configured to emit a sound waves configured to improve gas exchange in a patient's pulmonary system;
the first acoustic transducer residing within an acoustic housing comprising at least of one acoustic/sonic flue having an inlet and an outlet;
the first acoustic transducer positioned proximate to the inlet which is proportionately larger than the outlet;
said acoustic flue being in form of a linear, winding, or spiral-shaped gradually narrowing duct with any geometric cross sectional shape, configured to effectively focus and transmit the sound waves toward the outlet;
a ventilator operably connected to a respiratory gas conduit;
the gas conduit configured to be operably connected to a patient's airways;
the gas conduit configured to deliver the sound waves into the patient's airways, and
said ventilator being capable of pushing breathable gases into said airways 0-180 times per minute with pressure undulations of 0-100 cm H₂O.

2. The acoustic ventilation system of claim 1, wherein the sonic flue has a first cross-sectional dimension at the inlet of the flue and a second cross-sectional dimension at the outlet of the flue, wherein the first cross-sectional dimension is at least about 10% greater than the second cross-sectional dimension.

3. The acoustic ventilation system of claim 2, wherein the sonic flue has a first cross-sectional dimension at the outlet of the flue and a second cross-sectional dimension at the inlet of the flue, wherein the second cross-sectional dimension is at least about 50% greater than the first cross-sectional dimension.

4. The acoustic ventilation system of claim 2, wherein the sonic flue has a length in proportion to the flue's first cross-sectional dimension at the inlet of the flue and its second cross-sectional dimension at the outlet of the flue, wherein the second cross-sectional dimension is always smaller than the first cross-sectional dimension and the length is more than 10% of the amount of diameter of inlet squared divided by diameter of outlet squared, or L>0.1×D²/d².

5. The acoustic ventilation system of claim 2, wherein the sonic flue has a length in proportion to the flue's first cross-sectional dimension at the inlet of the flue and its second cross-sectional dimension at the outlet of the flue, wherein the second cross-sectional dimension is always smaller than the first cross-sectional dimension and the length is more than 50% of the amount of diameter of inlet squared divided by diameter of outlet squared, or L>0.5×D²/d².

6. The acoustic ventilation system of claim 1, wherein the first acoustic transducer is movable with respect to the channel to alter the frequency or resonance of the sonic flue.

7. The acoustic ventilation system of claim 2, wherein the first acoustic transducer is mounted on a guiderail and movable by a direct servomotor or a piston.

8. The acoustic ventilation system of claim 1, further comprising a second acoustic transducer configured for noise cancellation.

9. The acoustic ventilation system of claim 1, wherein the second acoustic transducer is in anti-phase with respect to the first acoustic transducer.

10. The acoustic ventilation system of claim 5, comprising a first sonic flue and a second sonic flue, wherein the second sonic flue housing the second acoustic transducer and transmitting anti-phase sonic vibrations with respect to the first flue.

11. The acoustic ventilation system of claim 1, further comprising an electronic noise cancellation device.

12. The acoustic ventilation system of claim 11, wherein the noise cancellation device comprises a muffler.

13. The acoustic ventilation system of claim 1, further comprising a double barrel endotracheal tube comprising an inspiratory flow lumen and an expiratory flow lumen, the endotracheal tube operably connected to a dual respiratory gas conduit system.

14. The acoustic ventilation system of claim 1, wherein the inspiratory flow lumen has a cross-sectional luminal area that is greater than a cross-sectional luminal area of the expiratory flow lumen.

15. The acoustic ventilation system of claim 1, wherein the inspiratory flow lumen has a cross-sectional luminal area that is less than a cross-sectional luminal area of the expiratory flow lumen.

16. The acoustic ventilation system of claim 1, wherein the inspiratory flow lumen has a cross-sectional luminal area that is equal to a cross-sectional luminal area of the expiratory flow lumen.

17. The acoustic ventilation system of claim 1, wherein the acoustic transducer comprises a piezoelectric crystal.

18. The acoustic ventilation system of claim 1, further comprising an acoustic signal analyzer configured to receive sonic information from sonic sensors mounted on patient's chest wall, analyze the information and accordingly adjust the machine's sonic vibrations in intensity and frequency along with FiO² and airway pressures through PFCC interface.

19. The acoustic ventilation system of claim 1, wherein the acoustic signal generator is configured to vary the signal in response to input from the acoustic signal analyzer.

20. The acoustic ventilation system of claim 1, wherein the ventilator comprises one or more ventilatory modes selected from the group consisting of: CMV, infrasonic oscillatory, sonic oscillatory, and ultrasonic oscillatory.

21. The acoustic ventilation system of claim 1, wherein the acoustic signal generator is configured to produce an acoustic signal having a frequency of between about 1 Hz to about 50 KHz.

22. The acoustic ventilation system of claim 1, wherein the acoustic signal generator is configured to produce an acoustic signal having a frequency of between about 50 KHz to about 2 MHz.

23. The acoustic ventilation system of claim 1 that further includes a method wherein especially designed recorded sound or music can improve respiration or enhance expectoration.

24. The acoustic ventilation system of claim 1 that further includes a method wherein a sonic adapter is used to modify regular ET tubes in order to make them usable for sonic ventilation.

25. The acoustic ventilation system of claim 1 that further includes a method, wherein the sonic flue can generate flow through use of bee wing valves.

26. The acoustic ventilation system of claim 1 that further includes a method wherein the acoustic ventilation further includes an adapter or a hub containing positive pressure and energizing inspiratory/expiratory gases with sonic vibrations and directing them in and out of patient's airways.

27. The acoustic ventilation system of claim 1 that further includes a method wherein an ET tube with ridged rim (spout rim) is employed to improve gas penetration into smaller airways.

28. An acoustic ventilation system, comprising:
an acoustic signal generator;
an acoustic signal transmitter operably connected to the acoustic signal generator;
the acoustic signal transmitter having an acoustic transducer configured to emit pressure oscillations from 1 Hz-2 MHz configured to improve gas exchange in a patient's pulmonary system;
the acoustic transducer residing within the inlet of an acoustic housing comprising at least one gradually narrowing channel with a closing angle less than 60 degrees and configured to transmit the sound waves to the channel's outlet which is proportionally smaller than the inlet;
a ventilator operably connected to a respiratory gas conduit;
the gas conduit configured to be operably connected to a patient's airway, and
the gas conduit configured to deliver the sound wave into the patient's airway along with tides of pressure changes from zero to 100 cm H2O and undulations of 0-180 per minute.

29. An acoustic ventilation system, comprising:
an acoustic signal generator;
an acoustic signal transmitter having an acoustic transducer configured to have a predetermined frequency and power to emit a sub-sonic;
and acoustic sound wave configured to affect respiratory gases sufficiently to ventilate an unintubated patient when placed in proximity to the patient.

30. A method of mechanically ventilating a patient to improve gas exchange, comprising:
generating an acoustic signal via an acoustic signal generator operably connected to an acoustic signal transmitter having an acoustic transducer;
ventilating a patient using a positive-pressure mechanical ventilator;
the ventilator delivering a flow of respiratory gas via a respiratory tubing circuit via a patient's airway into the lungs of the patient;
emitting the acoustic signal as a sound wave into the flow of respiratory gas such that the emitted signal improves gas exchange in the patient, and
delivering a therapeutic agent via the respiratory tubing into the airway and/or the lungs, such that the aerosolized therapeutic agent is distributed in the lungs more evenly compared to without the presence of the acoustic signal.

31. The method of claim 30, wherein the therapeutic agent comprises surfactant.

32. The method of claim 30, wherein the therapeutic agent comprises a respiratory aerosol such as a steroid, a beta-agonist, an anti-cholinergic agent, an alpha-agonist, a vasoactive agent, or an antibiotic.

33. The method of claim 30, wherein emitting the acoustic signal decreases oxygenation requirements of the patient.

34. The method of claim 30, wherein emitting the acoustic signal decreases positive pressure requirements of the patient and lowers pulmonary artery pressure.

35. The method of claim 30, wherein emitting the acoustic signal improves respiratory function in respiratory distress syndrome, cystic fibrosis, chronic obstructive pulmonary disease, obstructive sleep apnea, cerebral palsy, pulmonary embolism, bronchopulmonary dysplasia, interstitial lung disease, bronchiectasis, pneumonia, and asthma.

36. The method of claim 30, wherein emitting the acoustic signal enhances gas exchange, expectoration, and pulmonary perfusion.

37. The method of claim 30, wherein emitting the acoustic signal prevents alveolar collapse, consolidation, and atelectasis.

38. A method of improving gas exchange in an unintubated and breathing individual comprising:
generating an acoustic signal via an acoustic signal generator operably connected to an acoustic signal transmitter having an acoustic transducer;
emitting the acoustic signals as infrasonic and/or sonic waves such that the emitted waves improve gas exchange in the subject, wherein the subject may or may not simultaneously require positive pressure ventilation, and
said infrasonic and/or sonic waves delivered to the subject's airway directly via a facial mask, nasal prongs, or similar devices, or indirectly by vibrating the entire enclosure or room where the subject is located.

39. The method of claim 30, wherein emitting the acoustic signal improves respiratory function in respiratory distress syndrome, cystic fibrosis, chronic obstructive pulmonary disease, obstructive sleep apnea, cerebral palsy, pulmonary embolism, bronchopulmonary dysplasia, interstitial lung disease, bronchiectasis, pneumonia, and asthma.
